(19) United States
(12) Patent Application Publication
(10) Pub. No.: US 2013/0344140 A1
(43) Pub. Date: Dec. 26, 2013

(54) NOVEL PHARMACEUTICAL COMBINATIONS AND METHODS FOR TREATING CANCER

(71) Applicants: Chit Fang Cheok, Singapore (SG); David Lane, Singapore (SG)

(72) Inventors: Chit Fang Cheok, Singapore (SG); David Lane, Singapore (SG)

(73) Assignee: Agency for Science, Technology and Research, Singapore (SG)

(21) Appl. No.: 14/003,053

(22) PCT Filed: Mar. 4, 2011
(86) PCT No.: PCT/SG11/00086

§ 371 Date: Sep. 4, 2013

(51) Int. Cl. A61K 31/498; G01N 33/50; C12Q 1/68; A61K 31/496; G01N 33/68

(52) U.S. Cl. CPC A61K 31/498; A61K 31/496; G01N 33/6875; C12Q 1/6886; G01N 33/5011
USPC 424/451; 514/252.19; 424/490; 424/464; 435/7.1; 435/6.12; 435/6.11; 435/6.13; 435/7.4; 435/39

(57) ABSTRACT

Methods of selectively targeting a p53-deficient cancer cell, comprising administering to a patient suffering from cancer (i) a reversible cell cycle arrest-inducing agent for inducing cell cycle arrest in a p53-positive cell; and (ii) an aurora kinase inhibitor, wherein said reversible cell cycle arrest-inducing agent is administered prior to administration of said aurora kinase inhibitor, and pharmaceutical combinations, kits and oral dosage forms for the same.

### TECHNICAL FIELD

The present invention generally relates to methods for selectively targeting p53-deficient cancer cells without causing toxicity to p53-positive cells. The present invention also relates to pharmaceutical combinations, kits and oral dosage forms for use in such methods, as well as the use of such methods for identifying cancer patients for treatment with, and the use of such methods for treating duly identified cancer patients with, the pharmaceutical combinations, kits and oral dosage forms. In addition, the present invention relates to methods for screening and identifying compounds that selectively targets a p53-deficient cell.

### BACKGROUND

The tumor suppressor protein p53 is mutated in approximately 50% of all human tumors, and its pathways are inactivated in many of the remaining tumors due to amplification of its negative regulator, murine double minute 2 (MDM2), and/or loss of expression of alternate open reading frame (ARF). Tumor cells carrying mutations in the p53 gene typically lack normal regulation of cell cycle checkpoints, particularly the G1 checkpoint, and therefore divide in an unregulated and accelerated manner. Such p53 mutant cells continue to divide in the presence of chemotherapeutic agents, such as mitotic inhibitors, vinblastine, paclitaxel and S-phase inhibitors that are designed to target dividing cells, resulting in mitotic catastrophe and, ultimately, cell death. However, such chemotherapeutic agents also often result in the killing of normal, non-cancerous cells that carry wild-type p53 and have intact p53 pathways, and that are also undergoing cell division. This non-selective killing of cells, where both cancer cells carrying p53 mutations as well as non-cancerous cells carrying wild-type p53 are killed, limits the dosage of a chemotherapeutic agent that can be administered to a cancer patient to treat the cancer. As a result of such limitations, the optimal dosages that are required to effectively treat the cancer cannot be administered to the cancer patient without causing normal tissue toxicities.

A class of chemotherapeutic agents targeted at the later stages of the cell cycle from the G2/M check point through to the mitotic checkpoint and late mitosis is the aurora kinase inhibitors. Aurora kinases are key regulators of cell division, and regulate the segregation of chromatids during the mitosis phase of cell division to ensure proper distribution of genetic materials to daughter cells. Deregulation of aurora kinase activity can result in mitotic abnormality and genetic instability, which can lead to a monospindle phenotype, disruption of spindle assembly checkpoint (SAC), aberrant chromosomal segregation, cytokinesis failure, and endoreduplication, which ultimately results in polyploidy and cell death. As the expression level and kinase activity of aurora kinases are up-regulated in a wide variety of cancers, these kinases may provide useful targets in the development of chemotherapeutic drugs. However, as with other chemotherapeutic agents, successful treatment of cancer with aurora kinase inhibitors has also been hampered by dose-limiting toxicities, particularly the depletion of neutrophils in cancer patients.

There is a need to provide a method, as well as pharmaceutical compositions, kits and oral dosage forms, to selectively target p53-deficient cancer cells without compromising p53-positive non-cancerous cells that overcomes, or at least ameliorates, one or more of the disadvantages described above.

There is also a need to provide a method of screening to identify compounds that selectively targets a p53-deficient cancer cell or a p53-positive non-cancer cell for use as a chemotherapeutic agent.

### SUMMARY

According to a first aspect, there is provided a method of selectively targeting a p53-deficient cancer cell, comprising administering to a patient suffering from cancer:

(i) a reversible cell cycle arrest-inducing agent for inducing cell cycle arrest in a p53-positive cell; and

(ii) an aurora kinase inhibitor,

wherein the reversible cell cycle arrest-inducing agent is administered prior to administration of the aurora kinase inhibitor.

Advantageously, the administration of a reversible cell cycle arrest-inducing agent prior to administration of an aurora kinase inhibitor reversibly arrests the p53-positive cell in the G1 and G2 phase of the cell cycle. This prevents the p53-positive cell from entering mitosis and protects the p53-positive cell from the apoptotic effect of the aurora kinase inhibitor that is administered thereafter, while the p53-deficient cells that continue to undergo mitosis and cell division are selectively targeted. After the p53-deficient cancer cells are targeted, the temporarily arrested p53-positive non-cancer cells resume their cell cycle activities and undergo normal cell division. Advantageously, this provides for selective targeting of a p53-deficient cancer cell without compromising a p53-positive non-cancer cell, and allows treatment of cancer with aurora kinase inhibitors at its optimal dosages without, or with reduced, complications of dose-limiting toxicities.

According to a second aspect, there is provided a pharmaceutical combination for use in selectively targeting a p53-deficient cancer cell, in a patient suffering from cancer, comprising:

i) a reversible cell cycle arrest-inducing agent for inducing cell cycle arrest in a p53-positive cell; and

ii) an aurora kinase inhibitor,

wherein the reversible cell cycle arrest-inducing agent is provided in a form suitable for administration prior to administration of the aurora kinase inhibitor.

According to a third aspect, there is provided a method of identifying a cancer patient that is likely or not to respond to a therapy, comprising determining whether a cell or tissue sample isolated from the patient is p53-positive or p53 deficient, wherein determination that the cell or tissue sample is p53 deficient identifies the patient as likely to respond to the therapy, and wherein the therapy comprises administering to a patient:

(i) a reversible cell cycle arrest-inducing agent; and

(ii) an aurora kinase inhibitor,

and wherein the reversible cell cycle arrest-inducing agent is administered prior to administration of the aurora kinase inhibitor.

According to a fourth aspect, there is provided a method of treating a cancer patient identified as likely to respond to a therapy, wherein the patient is identified by a method comprising determining whether a cell or tissue sample isolated from the patient is p53-positive or p53 deficient, wherein determination that the cell or tissue sample is p53 deficient identifies the patient as likely to respond to the therapy, and wherein the method of treatment comprises administering to the patient:

(i) a reversible cell cycle arrest-inducing agent; and

(ii) an aurora kinase inhibitor,

wherein the reversible cell cycle arrest-inducing agent is administered prior to administration of the aurora kinase inhibitor.

According to a fifth aspect, there is provided a kit for use in selectively targeting p53-deficient cancer cells, in a patient suffering from cancer, the kit comprising:

i) a reversible cell cycle arrest-inducing agent for inducing cell cycle arrest in a p53-positive cell; and

ii) an aurora kinase inhibitor,

iii) instructions to administer the reversible cell cycle arrest-inducing agent prior to administration of the aurora kinase inhibitor.

According to a sixth aspect, there is provided a method for screening to identify a compound that selectively targets a first cell type or a second cell type, wherein the first cell type is a p53-deficient cell and wherein the second cell type is a p53-positive cell, and wherein the first cell type is labeled with a first detectable marker and wherein the second cell type is labeled with a second detectable marker, the method comprising:

(i) contacting the first and second cell types with the compound; and

(ii) determining the relative amounts of the first and second detectable markers,

wherein the first and second detectable marker are independently detectable and wherein a relative increase in the amount of the first marker in comparison to the amount of the second marker is indicative of a compound selectively targeting a p53-positive cell and wherein a relative increase in the amount of the second marker is indicative of a compound selectively targeting a p53-deficient cell.

Advantageously, the use of a first cell type and a second cell type enables the competitive growth selection factor to be accounted for, which is not the case if only a single cell type is used. By taking competitive growth selection into consideration, the disclosed method provides an accurate and physiologically relevant means of screening for candidate compounds based on their ability to selectively target a particular cell type. Competitive growth selection further leads to increased sensitivity and accuracy of assay and mimics the in vivo situation when there are cells of either genotype growing together in vivo.

### DEFINITIONS

The following words and terms used herein shall have the meaning indicated:

The term “selective targeting,” and grammatical variants thereof, as used herein refers to the killing, destroying, and/or inhibiting of a first cell or a first population of cells in preference to a second cell or a second population of cells, for example, by a margin of about 99:1 or above, about 95:1 or above, about 90:1 or above, about 85:1 or above, about 80:1 or above, about 75:1 or above, about 70:1 or above, about 65:1 or above, about 60:1 or above, about 55:1 or above, about 50:1 or above, about 45:1 or above, about 40:1 or above, about 35:1 or above, about 30:1 or above, about 25:1 or above, about 20:1 or above, about 15:1 or above, about 10:1 or above, about 9:1 or above, about 8:1 or above, about 7:1 or above, about 6:1 or above, about 5:1 or above, about 4:1 or above, about 3:1 or above, or about 2:1 or above. For example, a compound may selectively target a cell by binding to a first or target cell with greater affinity than to a second or non-target cell, for example, by about 10%, about 20%, about 30%, about 40%, about 50%, about 60%, about 70%, about 80%, about 90%, about 95%, or greater affinity relative to a non-target cell. The target cell may be a p53-deficient cell (e.g. a p53-negative cell or p53 mutant cell) and the non-target cell may be a p53-positive cell. Alternatively, the target cell may be a p53-positive cell and the non-target cell may be a p53-deficient cell (e.g. a p53-negative cell or p53 mutant cell).

The term “p53 deficient” as used herein refers to the lack of expression of the p53 gene due to mutation (e.g. rearrangement by mutation or viral targeting) or deletion of the gene. The term therefore includes “p53-negative,” that is, the p53−/− genotype, as well as “p53 mutant” harboring any detectable change in genetic material (e.g. DNA) or any process, mechanism, or result of such a change. This includes p53 gene mutations, in which the structure (e.g. DNA sequence) of the p53 gene is altered, any gene or DNA arising from any mutation process, and any expression product (e.g. p53 protein) expressed by a modified p53 gene or DNA sequence. In one embodiment of the present invention, the p53 deficient cell is a p53-negative cell. In a further embodiment of the present invention, the p53 deficient cell is a p53-mutant cell.

The term “p53 positive” as used herein relates to the genotype of a cell which possesses at least one copy of a gene encoding a functional p53 molecule. This includes cells having the p53+/+ genotype as well as the p53+/− genotype.

The term “contacting” in the context of contacting a cell type, e.g. a p53-deficient cell or a p53-positive cell, with a compound, e.g. a test compound, relates to bringing the compound and cell together by any means such that a compound that is selective for one cell type can bind to, target or interact with the cell either directly or indirectly. The contacting may be conducted in vitro (e.g. on cells that are in culture), ex vivo (e.g. on cells removed from and propagated outside a living organism), and/or in vivo (e.g. contacting within a living organism, such as a living human or other mammal, such as a mouse or rat).

The term “independently detectable” as used herein relates to labeling with detectable markers or moieties such that, where a first marker is used to label a first entity and a second marker is used to label a second entity, the first marker and second marker can each be detected. For example, the markers used may be able to emit light through different wavelengths, such that separate detection of each marker may be possible, for example, in different channels. Detection means include fluorescent activated cell sorting (FACS), chemiluminescence analysis or any other suitable means for detecting such markers.

The term “relative” when used with reference to the amount of a detectable marker relates to the amount of a first detectable marker compared to the amount of a second detectable marker. For example, a relative increase in the amount of the first detectable marker may signify a higher growth rate of a first cell type, for example in response to a test compound, and/or may signify the selective targeting of a second cell type by a test compound, such that the relative amount of the first cell type is increased in comparison to the amount of the second cell type.

The word “substantially” does not exclude “completely” e.g. a composition which is “substantially free” from Y may be completely free from Y. Where necessary, the word “substantially” may be omitted from the definition of the invention.

Unless specified otherwise, the terms “comprising” and “comprise”, and grammatical variants thereof, are intended to represent “open” or “inclusive” language such that they include recited elements but also permit inclusion of additional, unrecited elements.

As used herein, the term “about”, in the context of amounts or concentrations of components of the formulations, typically means +/−5% of the stated value, more typically +/−4% of the stated value, more typically +/−3% of the stated value, more typically, +/−2% of the stated value, even more typically +/−1% of the stated value, and even more typically +/−0.5% of the stated value.

Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Certain embodiments may also be described broadly and generically herein. Each of the narrower species and subgeneric groupings falling within the generic disclosure also form part of the disclosure. This includes the generic description of the embodiments with a proviso or negative limitation removing any subject matter from the genus, regardless of whether or not the excised material is specifically recited herein.

### Disclosure of Optional Embodiments

Exemplary, non-limiting embodiments of a method for selectively targeting p53-deficient cancer cells without causing toxicity to p53-positive cells, pharmaceutical combinations, kits and oral dosage forms for use therein, as well as a method for screening and identifying compounds that selectively target a p53-deficient cell or a p53-positive cell, will now be disclosed.

Methods for Selective Targeting of p53-Deficient Cancer Cells

The inventors of the present disclosure discovered that the administration of a reversible cell cycle arrest-inducing agent prior to administration of an aurora kinase inhibitor results in the selective targeting of p53-deficient cells and the reversible inhibition of proliferation in p53-positive cells.

Thus, the present disclosure relates to a method of selectively targeting p53-deficient cancer cells by administering to a patient suffering from cancer:

(i) a reversible cell cycle arrest-inducing agent for inducing cell cycle arrest in a p53-positive cell; and

(ii) an aurora kinase inhibitor, wherein the reversible cell cycle arrest-inducing agent is administered prior to administration of the aurora kinase inhibitor.

As used herein, the phrase “cell cycle arrest-inducing agent” refers to a compound of any type (e.g. non-selective or selective), including small molecule, antibody, antisense, small interfering RNA, or microRNA-based compounds, that induce cell cycle arrest in one or more cells. Induction of cell cycle arrest in one or more cells can be determined using routine techniques known in the art. For example, cell cycle arrest can be determined using any known methods in the art, for example a BrdU assay, flow cytometry, or microdensitometry.

In the context of this specification, the term “reversible cell cycle arrest” means that cell cycle arrest is not permanent but rather is short term and temporary. That is, the cell cycle is not permanently inactivated, but rather is arrested for a time sufficient to produce the desired effect and when the effects of the cell cycle arrest-inducing agent have diminished or ceased, the cell is available- to carry out its normal cellular division.

In one embodiment, the reversible cell cycle arrest-inducing agent is nutlin, a small-molecule inhibitor of the MDM2-p53 protein interaction. Nutlins act to inhibit the MDM2-p53 interaction by binding to the hydrophobic cleft of MDM2 and displacing p53. Nutlins comprise cis-imidazoline analogs that include nutlin-1, nutlin-2, nutlin-3 and nutlin-3a, also known as RG7112. Also contemplated for use in the disclosed methods are nutlin-like compounds and its enantiomers. Like nutlin, nutlin-like compounds are compounds capable of mimicking the binding of the helical region of p53 by interacting with the hydrophobic cleft of MDM2.

In a preferred embodiment, the reversible cell cycle arrest-inducing agent is nutlin-3.

In another preferred embodiment, the reversible cell cycle arrest-inducing agent is nutlin-3a.

An “aurora kinase inhibitor” refers to any molecule (e.g. non-selective or selective) which inactivates and/or down-regulates the activity of Aurora Kinase A, Aurora Kinase B and/or Aurora Kinase C (e.g. by interfering with interaction of the kinase with another molecule, such as its substrate), and/or decreases the protein level of Aurora Kinase A, Aurora Kinase B and/or Aurora Kinase C (e.g. by decreasing expression of the gene encoding the aurora kinase). The inhibitor can be a small molecule, an antisense nucleic acid, a ribozyme, an antibody, a dominant negative mutant of the kinase, a small interfering RNA, a microRNA-based compound, or a phosphatase.

The aurora kinase inhibitor may be a direct inhibitor such that it interacts with the kinase or binding partner thereof or with a nucleic acid encoding the kinase. Alternatively, the aurora kinase inhibitor may be an indirect inhibitor which interacts upstream or downstream of the kinase in the regulatory pathway and which does not interact with the kinase or binding partner thereof or with a nucleic acid encoding the kinase.

In one embodiment, the aurora kinase inhibitor is an Aurora Kinase A inhibitor. Exemplary Aurora Kinase A inhibitors include, but are not limited to, PHA-739358, MLN-8054, R-763, JNJ-7706621, MP-529 and MP-235.

In another embodiment, the aurora kinase inhibitor is an Aurora Kinase B inhibitor. A number of Aurora Kinase B inhibitors are known to inhibit at least one of histone H3 phosphorylation or cell division, and to induce apoptosis in at least one cell system (such as an acute myeloid leukemia cell line, a, primary acute myeloid leukemia culture, etc.). Exemplary Aurora Kinase B inhibitors include, but are not limited to, VX-680/MK0457, AZD1152, ZM447439 and Hesperadin. Aurora kinase A is also known as Aurora A kinase or Aurora A; Aurora kinase B is also known as Aurora B kinase or Aurora B; Aurora kinase C is also known as Aurora C kinase or Aurora C, and these terms are used interchangeably herein.

VX-680, also referred to as MK0457 and VX-680/MK0457, is a cyclopropane carboxylic acid of {4-[4-(4-methyl-piperazin-1-yl)-6-(5-methyl-2H-pyrazol-3-ylamino)-pyrimidin-2-ylsulphanyl]-phenyl}-amide and inhibits Aurora A, Aurora B and Aurora C.

AZD1152, also known as 2-[[3-({4-[(5-{2-[(3-Fluorophenyl)amino]-2-oxoethyl}-1H-pyrazol-3-yl)amino]-quinazolin-7-yl}oxy)propyl](ethyl) amino]ethyl dihydrogen phosphate, is a prodrug of a pyrazoloquinazoline Aurora Kinase inhibitor (AZD1152-hydroxyquinazoline pyrazol anilide (HQPA)) and is converted rapidly in plasma to the active AZD1152-HQPA, a highly potent and selective inhibitor of Aurora B.

ZM447439, also known as 4-(4-(N-benzoylamino)anilino)-6-methoxy-7-(3-(1-morpholino) propoxy)quinazoline, is a quinazoline derivative, and inhibits Aurora A and Aurora B.

Hesperadin is an indolinone and inhibits Aurora B.

In yet another embodiment, the aurora kinase inhibitor is an Aurora Kinase C inhibitor. Exemplary Aurora Kinase C inhibitors include, but are not limited to, AZD1152 and VX-680/MK-0457.

In a preferred embodiment, the aurora kinase inhibitor is an Aurora Kinase B inhibitor and/or an Aurora Kinase C inhibitor.

In a most preferred embodiment, the aurora kinase inhibitor is VX-680.

In one embodiment, only one aurora kinase inhibitor is administered after administration of the reversible cell cycle arrest-inducing agent. In another embodiment, a combination of more than one, for example 2, 3, 4, 5, 6, 7, 8, 9, 10, etc., aurora kinases are administered after administration of the reversible cell cycle arrest-inducing agent.

In one embodiment, the reversible cell cycle arrest-inducing agent is nutlin-3 and the aurora kinase inhibitor is VX-680.

In one embodiment, p53 deficient cancer cells are selectively targeted using the disclosed methods. Such cancer cells include, but are not limited to, leukemia cells; lymphoma cells; myeloma cells; skin cancer cells such as basal cell carcinoma (BCC) cells, squamous cell carcinoma (SCC) cells or melanoma cells; breast cancer cells; head and neck cancer cells, such as brain cancer cells; colorectal cancer cells; colon cancer cells; rectal cancer cells; lung cancer cells, such as non small cell lung cancer cells; ovarian cancer cells; renal cancer cells; prostate cancer cells; liver cancer cells; and HPV-associated cancer cells such as cervical cancer cells.

In the disclosed method, the reversible cell cycle arrest-inducing agent is to be administered prior to administration of the aurora kinase inhibitor. For example, the reversible cell cycle arrest-inducing agent may be administered about 12 to about 72 hours, about 16 to about 60 hours, about 18 to about 48 hours, or about 24 to about 36 hours prior to administration of the aurora kinase inhibitor. Preferably, the reversible cell cycle arrest-inducing agent is administered about 12, about 16, about 18, about 24, about 36, about 48, about 72 hours prior to administration of the aurora kinase inhibitor.

The reversible cell cycle arrest-inducing agent and an aurora kinase inhibitor may be administered in a therapeutically effective amount for use in the methods disclosed herein. The term “therapeutically effective amount” as used herein includes within its meaning a sufficient but non-toxic amount of the reversible cell cycle arrest-inducing agent and/or an aurora kinase inhibitor to provide the desired therapeutic effect. The exact amount required will vary from subject to subject depending on factors such as the species being treated, the age and general condition of the subject, the severity of the condition being treated, the particular agent being administered, the mode of administration, and so forth. For any given case, an appropriate “effective amount” may be determined by one of ordinary skill in the art using only routine experimentation.

### Pharmaceutical Combinations

The present disclosure also relates to pharmaceutical combinations that are useful in treating patients suffering from cancer by selectively targeting p53-deficient cancer cells in the patient. As mentioned previously herein, the inventors of the present disclosure discovered that the induction of reversible cell cycle arrest in p53-positive cells protects these cells from the effects of a subsequently administered aurora kinase inhibitor.

Thus, the present disclosure relates to a pharmaceutical combination of therapeutic agents (namely, a combination therapy) that comprises:

i) a reversible cell cycle arrest-inducing agent for inducing cell cycle arrest in a p53-positive cell; and

ii) an aurora kinase inhibitor.

In one embodiment, the reversible cell cycle arrest-inducing agent is provided in a form suitable for administration prior to administration of the aurora kinase inhibitor.

In one embodiment, the reversible cell cycle arrest-inducing agent is nutlin-3 and the aurora kinase inhibitor is VX-680.

In one embodiment in which administration is in vitro, the range of nutlin to be administered is from about 5 uM to about 20 uM, and the range of VX-680 to be administered is from about 100 nM to about 10 uM.

The disclosed pharmaceutical combination can be administered to a patient suffering from any type of tumor, cancer, malignancy or combinations thereof. For example, the pharmaceutical combinations can be used to treat a patient suffering from colorectal cancer, fibrosarcoma, myxosarcoma, liposarcoma, chondrosarcoma, osteogenic sarcoma, chordoma, angiosarcoma, endotheliosarcoma, lymphangiosarcoma, lymphangio-endotheliosarcoma, synovioma, mesothelioma, Ewing's tumor, leiomyosarcoma, rhabdomyosarcoma, colon carcinoma, gastic cancer, pancreatic cancer, breast cancer, ovarian cancer, prostate cancer, squamous cell carcinoma of the head and neck, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, liver metastases, bile duct carcinoma, choriocarcinoma, seminoma, embryonal carcinoma, thyroid carcinoma such as anaplastic thyroid cancer, Wilms' tumor, cervical cancer, testicular tumor, lung cancer, small cell cancer of the lung, non-small cell cancer of the lung, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, and retinoblastoma.

Suitable pharmaceutical compositions comprising the combination of a reversible cell cycle arrest-inducing agent as disclosed herein with an aurora kinase inhibitor as disclosed herein may be prepared according to methods which are known to those of ordinary skill in the art and accordingly may include a pharmaceutically acceptable carrier.

The term “carrier” refers, for example to a diluent, adjuvant, excipient, auxilliary agent or vehicle with which the disclosed pharmaceutical combination can be administered. Such pharmaceutically acceptable carriers can be sterile liquids, such as water and oils, including those of petroleum, animal, vegetable or synthetic origin, such as peanut oil, soybean oil, mineral oil, sesame oil and the like. Preferably, particularly for injectable solutions, water or aqueous saline solutions and aqueous dextrose and glycerol solutions are employed as carriers. The pharmaceutical combinations disclosed herein can also include diluents of various buffer content (e.g. Tris-HCl, acetate, phosphate), pH and ionic strength; additives such as detergents and solubilizing agents (e.g. Tween 80, Polysorbate 80), anti-oxidants (e.g. ascorbic acid, sodium metabisulfite), preservatives (e.g. Thimersol, benzyl alcohol) and bulking substances (e.g. lactose, mannitol); and can be incorporated into particulate preparations of polymeric compounds such as polylactic acid, polyglycolic acid, etc., or into liposomes. Liposomes are generally derived from phospholipids or other lipid substances, and are formed by mono- or multi-lamellar hydrated liquid crystals that are dispersed in an aqueous medium. Any non-toxic, physiologically acceptable and metabolisable lipid capable of forming liposomes can be used. The compositions in liposome form may contain stabilisers, preservatives, excipients and the like. The preferred lipids are the phospholipids and the phosphatidyl cholines 20″ (lecithins), both natural and synthetic. Methods to form liposomes are known in the art, and in relation to this specific reference is made to: Prescott, Ed., Methods in Cell Biology, Volume XIV, Academic Press, New York, N.Y. (1976), p. 33 et seq., the contents of which is incorporated herein by reference.

Other suitable pharmaceutical carriers are described in “Remington's Pharmaceutical Sciences” 15th ed., Mack Publishing Company, Easton, Pa., hereby incorporated by reference herein.

The carriers, diluents and/or adjuvants must be “pharmaceutically acceptable” in terms of being compatible with the other ingredients of the composition, and not deleterious to the recipient thereof.

Typically, the carrier or carriers will form from 10% to 99.9% by weight of the compositions. Such compositions may influence the physical state, stability, rate of in vivo release, and rate of in vivo clearance of components of the disclosed pharmaceutical combinations. A pharmaceutical combination as disclosed herein can be prepared, for example, in liquid form, or can be in dried powder, such as lyophilized form.

The disclosed compositions can be administered by standard routes such as parenteral (e.g. intravenous, intraspinal, subcutaneous or intramuscular), oral, nasal, intra- or trans-dermal, rectal or topical route. Preferably, administration is oral or parenteral, e.g. via intravenous injection or by injection into the tumor(s) being treated or into tissues surrounding the tumor(s). Depending on the route of administration, the pharmaceutical combination may be coated with a material to protect the constituent compounds from the action of enzymes, acids and other natural conditions which may inactivate the therapeutic activity of the compounds.

In one embodiment, the disclosed compositions are in a form suitable for administration by injection, in the form of a formulation suitable for oral ingestion (such as capsules, tablets, caplets, elixirs, for example), or in a form suitable for parenteral administration, that is, subcutaneous, intramuscular or intravenous injection.

In one embodiment, the disclosed pharmaceutical combination can be delivered in a controlled release system, such as using an intravenous infusion, an implantable osmotic pump, a transdermal patch, liposomes, or other modes of administration. In one embodiment, a pump is used. In another embodiment, polymeric materials are used. In yet another embodiment, a controlled release system can be placed in proximity of the target tissues of the subject, thus requiring only a fraction of the systemic dose. For example, a controlled release device can be introduced into a subject in proximity of the site of a tumor.

In one embodiment, the disclosed pharmaceutical combination is a dosage form formulated as enterically coated granules, tablets or capsules suitable for oral administration. Enteric formulations, or enterically coated formulations, as used herein, may refer to a composition that is coated with a substance that remains intact in the stomach, but dissolves and releases the formulated composition once the small intestine is reached. For example, in one embodiment, the disclosed pharmaceutical combinations are formulated as delayed release formulations, for example, a form in which the composition has an enteric coating allowing release of the composition as required in a gradual or delayed release manner.

In one embodiment, there is provided an oral dosage form comprising:

(i) a first composition comprising a reversible cell cycle arrest-inducing agent for inducing cell cycle arrest in a p53-positive cell; and

(ii) a second composition comprising an aurora kinase inhibitor,

wherein said first composition is formulated for immediate release on contact with aqueous media and wherein said second composition is formulated for modified release on contact with aqueous media.

The term “immediate release” as used herein relates to a dosage form that delivers the entirety of its drug content as quickly as possible after administration. Immediate release may be provided for by way of an appropriate pharmaceutically acceptable carrier or diluent, which carrier or diluent does not prolong, to an appreciable extent, the rate of drug release and/or absorption. Such formulations may release at least 70% (preferably at least 80%, 90%, 95%, or 99%) of active ingredient within 4 hours, such as within 3 hours, preferably 2 hours, more preferably within 1.5 hours, and most preferably within an hour (such as within 30 minutes), of administration, whether this be oral or parenteral.

The term “modified release” as used herein relates to a dosage form whose drug-release characteristics of time course and/or location are selected to achieve therapeutic or convenience objectives. Modified release dosage forms include dosage forms commonly known in the art as, delayed, sustained, extended, targeted, prolonged, pulsatile, zero-order, constant rate, and controlled.

In one embodiment, the modified release comprises delayed and/or sustained release. For example, the modified release of drug substance from a pharmaceutical formulation may be at a slower rate than from an immediate release formulation.

The term “dosage form” as used herein relates to tablets, capsules, mini-tablets, caplets, uncoated micro-particles, micro-particles coated with at least one release-retarding coating, micro-particles coated with at least one delayed-release coating, and any combination thereof. These dosage forms may also be modified release, osmosis modified-release, erosion modified-release, diffusion modified-release, matrix cores, matrix cores coated with release-slowing coatings, enteric coated, dosage forms surrounded by slow or delayed release coatings, gastroretentive dosage forms, or muco-adhesive dosage forms.

In one embodiment, the first and second compositions are multiparticulates. Multiparticulates are dosage forms that comprise a multiplicity of particles whose totality represents the intended therapeutically useful dose of a drug. When taken orally, multiparticulates disperse freely in the gastrointestinal tract to maximize absorption and minimize side effects.

The multiparticulates comprising the first composition may be uncoated and the multiparticulates comprising the second composition may have an enteric coat.

In one embodiment, the second composition is coated with an enteric coat.

In one embodiment, the multiparticulates are filled into a capsule.

In one embodiment, the oral dosage form takes the form of a tablet in which the first and second compositions are arranged in two or more separate layers.

### Method of Identifying Suitable Patients

The inventors of the present disclosure further found that nutlin only exerted its protective effect on cells that were p53-positive. This enables selective targeting of p53-deficient cancer cells in a cancer patient by aurora kinase inhibitors subsequently administered to the patient. Accordingly, the present disclosure further provides a method of identifying a cancer patient that is likely or not to respond to a therapy, comprising determining whether a cell or tissue sample isolated from the patient is p53-positive or p53 deficient, for example p53 negative, wherein determination that the cell or tissue sample is p53 deficient identifies the patient as likely to respond to the therapy, and wherein the therapy comprises administering to a patient:

(i) a reversible cell cycle arrest-inducing agent; and

(ii) an aurora kinase inhibitor,

and wherein the reversible cell cycle arrest-inducing agent is administered prior to administration of the aurora kinase inhibitor.

A cancer patient may be determined to be likely to respond to the therapy if a patient derived cell or tissue sample is determined to be p53 deficient, for example, the cell or tissue sample has tested negative for p53, or lacks expression of the p53 gene due to mutation or deletion of the p53 gene. A person having a genetic predisposition to developing a mutation or deletion of the p53 gene, such as a person having a familial disposition to cancer, may also be considered as a patient who is likely to respond to therapy.

As used herein, the term “sample” or “patient derived sample” includes tissues, cells, body fluids and isolates thereof etc., isolated from a subject, as well as tissues, cells and fluids etc. present within a subject (i.e. the sample is in vivo). By “isolated,” it is meant that the sample is substantially or essentially free from components that normally accompany it in its native environment. Examples of samples include: whole blood, blood fluids (e.g. serum and plasm), lymph and cystic fluids, sputum, stool, tears, mucus, hair, skin, ascitic fluid, cystic fluid, urine, nipple exudates, nipple aspirates, sections of tissues such as biopsy and autopsy samples, frozen sections taken for histologic purposes, archival samples, explants and primary and/or transformed cell cultures derived from patient tissues etc.

In the disclosed method, the sample may be a cell or tissue sample, such as a patient-derived tissue sample of any type or on a derivative or extract thereof, including peripheral blood, tumor or suspected tumor tissues (including fresh frozen and fixed paraffin-embedded tissue), cell isolates such as circulating epithelial cells separated or identified in a blood sample, lymph node tissue, bone marrow and fine-needle aspirates.

The sample may be untreated, treated, diluted or concentrated from a patient. For example, the sample can be used directly as obtained from the source, or following a pre-treatment to modify the character of the sample. A test sample can be pre-treated prior to use by, for example, preparing plasma or serum from blood, disrupting cells or viral particles, preparing liquids from solid materials, diluting viscous fluids, filtering liquids, adding reagents, purifying nucleic acids, etc. The sample on which the disclosed method is performed may therefore also comprise an extract from a cell, chromosome, organelle, or membrane isolated from a cell; genomic DNA, RNA, or cDNA, in solution or bound to a substrate.

The sample may be obtained at one or more time points. Other than determining whether the sample is p53-positive or p53-deficient, expression levels of the p53 gene in the sample may optionally be compared with a “control” sample. In one embodiment, the “control” sample may be a matched tissue sample derived from the patient. One or more control samples may be employed.

The term “patient” refers to patients that are human or other mammals and includes any individual it is desired to examine or treat using the disclosed methods. However, it will be understood that “patient” does not imply that symptoms are present. Suitable mammals that fall within the scope of the invention include, but are not restricted to, primates, livestock animals (e.g. sheep, cows, horses, donkeys, pigs), laboratory test animals (e.g. rabbits, mice, rats, guinea pigs, hamsters), companion animals (e.g. cats, dogs) and captive wild animals (e.g. foxes, deer, dingoes).

The disclosed methods can be used with samples obtained from a patient suffering from colorectal cancer, fibrosarcoma, myxosarcoma, liposarcoma, chondrosarcoma, osteogenic sarcoma, chordoma, angiosarcoma, endotheliosarcoma, lymphangiosarcoma, lymphangio-endotheliosarcoma, synovioma, mesothelioma, Ewing's tumor, leiomyosarcoma, rhabdomyosarcoma, colon carcinoma, gastic cancer, pancreatic cancer, breast cancer, ovarian cancer, prostate cancer, squamous cell carcinoma of the head and neck, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, liver metastases, bile duct carcinoma, choriocarcinoma, seminoma, embryonal carcinoma, thyroid carcinoma such as anaplastic thyroid cancer, Wilms' tumor, cervical cancer, testicular tumor, lung cancer, small cell cancer of the lung, non-small cell cancer of the lung, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, or retinoblastoma.

To determine whether the sample is p53-positive or p53-deficient, one or more techniques known in the art may be used. For example, P53 expression can be determined by any one of immunohistochemistry (IHC), direct deep clonal sequencing flow cytometric analysis, PCR analysis, and/or any other routine methods known in the art.

### Method of Treatment

The inventors' findings that the induction of reversible cell cycle arrest in p53-positive cells protects these cells from the effects of a subsequently administered aurora kinase inhibitor further provides a method of treating a cancer patient. Preferably, the treatment method is performed on a patient who has been identified using the identification method disclosed herein as being likely to respond to the therapy, that is, a patient whose cell or tissue sample has been determined to be p53-deficient. The method of treatment comprises administering to the patient a pharmaceutical combination as disclosed herein comprising a reversible cell cycle arrest-inducing agent and an aurora kinase inhibitor, wherein the reversible cell, cycle arrest-inducing agent is administered prior to administration of the aurora kinase inhibitor.

The terms “treat,” “treatment,” and grammatical variants thereof, refer to both therapeutic treatment and prophylactic or preventative measures, wherein the object is to prevent or slow down (lessen) an undesired physiological condition, disorder or disease or obtain beneficial or desired clinical results. Such beneficial or desired clinical results include, but are not limited to, alleviation of symptoms; diminishment of extent of condition, disorder or disease; stabilized (i.e. not worsening) state of condition, disorder or disease; delay or slowing of condition, disorder or disease progression; amelioration of the condition, disorder or disease state, remission (whether partial or total), whether detectable or undetectable; or enhancement or improvement of condition, disorder or disease. Treatment includes eliciting a cellular response that is clinically significant, without excessive levels of side effects. Treatment also includes prolonging survival as compared to expected survival if not receiving treatment.

Typically, in therapeutic applications, the treatment would be administered as required. Further, it will be apparent to one of ordinary skill in the art that the optimal quantity and spacing of individual dosages will be determined by the nature and extent of the disease state being treated, the form, route and site of administration, the nature of the particular individual being treated, and whether or not another drug is co-administered. Also, such optimum conditions can be determined by conventional techniques.

Single or multiple administrations of the disclosed pharmaceutical compositions may be carried out. One skilled in the art would be able, by routine experimentation or using conventional course of treatment determination tests, to determine effective, non-toxic dosage levels of the disclosed pharmaceutical compositions and its constituent compounds, and an administration pattern (such as the number of doses given per day for a defined number of days) which would be suitable for treating the cancer.

Generally, an effective dosage per 24 hours may be in the range of about 0.0001 mg to about 1000 mg per kg body weight; suitably, about 0.001 mg to about 750 mg per kg body weight; about 0.01 mg to about 500 mg per kg body weight; about 0.1 mg to about 500 mg per kg body weight; about 0.1 mg to about 250 mg per kg body weight; or about 1.0 mg to about 250 mg per kg body weight. More suitably, an effective dosage per 24 hours may be in the range of about 1.0 mg to about 200 mg per kg body weight; about 1.0 mg to about 100 mg per kg body weight; about 1.0 mg to about 50 mg per kg body weight; about 1.0 mg to about 25 mg per kg body weight; about 5.0 mg to about 50 mg per kg body weight; about 5.0 mg to about 20 mg per kg body weight; or about 5.0 mg to about 15 mg per kg body weight.

Alternatively, an effective dosage may be up to about 500 mg per kg body weight. For example, generally, an effective dosage is expected to be in the range of about 25 to about 500 mg per kg body weight, about 25 to about 350 mg per kg body weight, about 25 to about 300 mg per kg body weight, about 25 to about 250 mg per kg body weight, about 50 to about 250 mg per kg body weight, and about 75 to about 150 mg per kg body weight.

The effective daily dosage may be administered in a single dose or in divided doses. Single dose compositions contain these amounts or a combination of submultiples thereof.

For monitoring patient response, determination of the p53 status or expression in a cell or tissue sample isolated from a patient can be performed at the initiation of therapy (for example, at the time of administration of at least one reversible cell cycle arrest-inducing agent or after administration of at least one reversible cell cycle arrest-inducing agent but prior to the administration of at least one aurora kinase inhibitor) to establish the baseline levels of p53 expression and/or mutation(s) in the cell or tissue sample, for example, the percent of total cells or number of cells showing at least p53 deficient expression in the sample. The same cell or tissue may then be sampled and assayed and the amount of p53 deficient expression, for example the number of p53 mutations, are then compared to the baseline or predetermined level. In one embodiment, where the number of p53 mutations remains the same or decreases, the therapy is likely being effective and can be continued. Where significant increase in p53 mutations over baseline level occurs, the patient may not be responding or may have developed resistance to continued treatment using the reversible cell cycle arrest-inducing agent and/or the aurora kinase inhibitor.

### Kits

The present disclosure also contemplates a variety of kits for use in the disclosed methods. The kits comprise:

i) a reversible cell cycle arrest-inducing agent for inducing cell cycle arrest in a p53-positive cell;

ii) an aurora kinase inhibitor; and

iii) instructions to administer the reversible cell cycle arrest-inducing agent prior to administration of the aurora kinase inhibitor.

The instructions may be provided in paper form or in computer-readable form, such as a disc, CD, DVD or the like.

The kits may optionally include quality control reagents, such as sensitivity panels, calibrators, and positive controls. The kits can further incorporate a detectable label, such as a fluorophore, radioactive moiety, enzyme, biotin/avidin label, chromophore, chemiluminescent label, or the like, or the kits may include reagents for labeling the nucleic acid primers, the nucleic acid probes or the nucleic acid primers and nucleic acid probes for detecting the presence or absence of at least one mutation as described herein. The primers and/or probes, calibrators and/or controls can be provided in separate containers or pre-dispensed into an appropriate assay format, for example, into microtiter plates.

The kits can optionally include other reagents required to conduct a diagnostic assay or facilitate quality control evaluations, such as buffers, salts, enzymes, enzyme co-factors, substrates, detection reagents, and the like. Other components, such as buffers and solutions for the isolation and/or treatment of a test sample (e.g., pretreatment reagents), may also be included in the kit. The kit may additionally include one or more other controls. One or more of the components of the kit may be lyophilized and the kit may further comprise reagents suitable for the reconstitution of the lyophilized components.

The various components of the kit optionally are provided in suitable containers. As indicated above, one or more of the containers may be a microtiter plate. The kit further can include containers for holding or storing a sample (e.g., a container or cartridge for a blood or urine sample). Where appropriate, the kit may also optionally contain reaction vessels, mixing vessels and other components that facilitate the preparation of reagents or the test sample. The kit may also include one or more instruments for assisting with obtaining a test sample, such as a syringe, pipette, forceps, measured spoon, or the like.

### Screening Assays

The inventors additionally found that the labeling of a first type of cells that may be selectively targeted by a test compound with a first detectable marker and a second type of cells that may be protected from the effect of the test compound with a second detectable marker provides a sensitive and accurate assay for screening compounds that can be used to treat cancer without causing normal tissue toxicities. The disclosure therefore provides a method for screening to identify a compound that may selectively target a first cell type or a second cell type, wherein the first cell type is a p53-deficient cell and wherein the second cell type is a p53-positive cell, and wherein the first cell type is labeled with a first detectable marker and wherein the second cell type is labeled with a second detectable marker, the method comprising:

(i) contacting the first and second cell types with the compound; and

(ii) determining the relative amounts of the first and second detectable markers,

wherein the first and second detectable marker are independently detectable and wherein a relative increase in the amount of the first marker in comparison to the amount of the second marker is indicative of a compound selectively targeting a p53-positive cell and wherein a relative increase in the amount of the second marker is indicative of a compound selectively targeting a p53-deficient cell.

Any compound, known or unknown, may be screened using the disclosed method to determine the ability of the compound to selectively target p53-deficient cancer cells but not p53-positive non-cancer cells. The compound may be organic molecules, such as small organic compounds (i.e. small molecules, preferably having a molecular weight of more than 100 and less than about 2,500 daltons), chemical compounds, chemical combinations and salts thereof, or natural products such as plant extracts or materials obtained from fermentation broths. Candidate compounds preferably comprise functional groups necessary for structural interaction with proteins, particularly hydrogen bonding, and typically include at least an amine, carbonyl, hydroxyl or carboxyl group, preferably at least two of the functional chemical groups. The candidate compounds may comprise cyclical carbon or heterocyclic structures and/or aromatic or polyaromatic structures substituted with one or more of the above functional groups. Candidate compounds may also be biomolecules such as proteins, peptides or fragments thereof, nucleic acids and oligonucleotides, carbohydrates (e.g. saccharides), phospholipids and other lipid derivatives (e.g. fatty acids, steroids, etc.), prostaglandins and related arachadonic acid derivatives, purines, pyrimidines, and derivatives, structural analogs, or combinations thereof.

Candidate compounds may be obtained from a wide variety of synthetic or natural sources, such as libraries of synthetic or natural compounds. For example, libraries of natural compounds in the form of bacterial, fungal, plant and animal extracts are available or readily produced. Various means are also available for random and directed synthesis of a wide variety of organic compounds and biomolecules, including expression of randomized oligonucleotides. Such natural or synthetically produced libraries and compounds can furthermore be modified through conventional chemical, physical and biochemical means.

In one embodiment, the first cell type is a p53-deficient cell type, for example a cancer cell type selected from the group consisting of leukemia cells; lymphoma cells; myeloma cells; skin cancer cells such as basal cell carcinoma (BCC) cells, squamous cell carcinoma (SCC) cells or melanoma cells; breast cancer cells; head and neck cancer cells, such as brain cancer cells; colorectal cancer cells; colon cancer cells; rectal cancer cells; lung cancer cells, such as non small cell lung cancer cells; ovarian cancer cells; renal cancer cells; prostate cancer cells; liver cancer cells; and HPV-associated cancer cells such as cervical cancer cells. The cell type may have a p53−/− genotype.

In one embodiment, the second cell type is a p53-positive cell type, for example a non-cancer cell having the p53+/+ genotype or a non-cancer cell having the p53+/− genotype.

In one embodiment, the first cell type is labeled with a first detectable marker and the second cell type is labeled with a second detectable marker.

The term “labeled”, with regard to the first and/or second cell type is intended to encompass direct labeling of the first and/or second cell type by coupling (i.e., physically linking) a detectable substance to the first and/or second cell type, as well as indirect labeling of the first and/or second cell type by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin.

The “detectable marker” as used herein refers to a reporter molecule or enzyme that is capable of generating a measurable signal (e.g. a fluorescent signal, a radioactive signal, a bioluminescent signal, etc.) and may be covalently or non-covalently joined to a polynucleotide or polypeptide. Detectable markers suitable for use in the disclosed method include, but are not limited to, fluorescent compounds, radioisotope compounds, non-radioisotope compounds, bioluminescent compounds, chemiluminescent compounds, metal chelator compounds, chromogenic compounds, X-radiographic compounds, and enzymes. Exemplary fluorescent compounds that may be used include, but are not limited to, Green Fluorescent Protein (GFP), Red Fluorescent Protein (RFP), fluorescein, rhodamine, phycoerytherin, phycocyanin, allophycocyanin, o-phthaldehyde and fluorescamine. Exemplary radioisotope compounds that may be used include, but are not limited to, molybdenum-99, thallium-204, calcium-45, cobalt-60 and/or thulium-170.

Exemplary bioluminescent compounds that may be used include, but are not limited to, firefly luciferin, Latia luciferin, Dinoflagellate luciferin, coelenterazine, and/or vargulin.

Exemplary chemiluminescent compounds that may be used include, but are not limited to, Luminol, Cyalume, Oxalyl chloride and/or Pyrogallol.

Exemplary metal chelator compounds that may be used include, but are not limited to, ethylenediamine tetraacetic acid (EDTA), cyclohexane diaminetetraacetate (CDTA), diethylenetriamine pentaacetic acid (DPTA), tetraazacyclododecanetetraacetic acid (DOTA), tetraazacyclotetradecanetetraacetic acid (TETA), and desferrioximine, or chelator analogs thereof.

Exemplary chromogenic compounds that may be used include, but are not limited to, hide powder azure, S 2423, indophane blue, and/or blue ciello.

Exemplary X-radiographic compounds that may be used include, but are not limited to, Iridium 192, Cobalt 60 and/or Cesium 137.

Exemplary enzymes that may be used include, but are not limited to, horse radish peroxidase, firefly luciferase, soybean peroxidase, and/or beta-Galactosidase.

In a preferred embodiment, the first cell type is labeled with RFP and the second cell type is labeled with GFP.

In one embodiment, the first and second cell types are contacted with the candidate compound in vitro. When the contact is in vitro, the first and second cell types may be in a cell culture. In one embodiment, the culture medium includes the necessary nutrients and co-factors required for in vitro growth and development of the first and second cell types. Preferably the culture medium contains at least water, salts, nutrients, essential amino acids, vitamins and hormones, and may also include one or more growth factors. A variety of suitable culture media is commercially available, for example Earle's media, Ham's F10 media and human tubal fluid (HTF) media.

The culture medium optionally includes a pre-determined amount of the candidate compound prior to seeding the culture medium with the first or second cell types. Alternatively, the candidate compound is added to the culture medium at a later time after culturing the first and second cell types for a pre-determined period of time.

In one embodiment, the first and second cell types are contacted with the candidate compound ex vivo. For example, the first and second cell types may be contacted with the candidate compound in or on living cells or tissues that have been taken from an organism and placed in an artificial environment outside the organism, for example, cultured in a laboratory apparatus typically under sterile conditions with no alterations for up to about 24 hours.

In one embodiment, the first and second cell types are contacted with the candidate compound in vivo. For example, the first and second cell types may be within a patient (e.g. a patient suffering from or suspected of having cancer) and the candidate compound is administered to the patient. Alternatively, the first and second cell types may be within a non-human animal model system. The animal model system may be a transgenic animal model system. The animal may be a mammal, such as a mouse, a rat, a dog, a pig, a goat, a cow, etc.

After contacting the first cell type and the second cell type with the candidate compound, the relative amounts of the first and second detectable markers bound to the first and second cell type, respectively, may be determined using techniques such as fluorescence-activated cell sorting (FACS), and/or quantitative imaging microscopy.

A relative increase in the amount of the first marker in comparison to the amount of the second marker indicates that a candidate compound selectively targets a p53-positive cell. Conversely, a relative increase in the amount of the second marker indicates that a candidate compound selectively targets a p53-deficient cell.

### BRIEF DESCRIPTION OF DRAWINGS, TABLES AND SEQUENCES

The accompanying drawings illustrate a disclosed embodiment and serves to explain the principles of the disclosed embodiment. It is to be understood, however, that the drawings are designed for purposes of illustration only, and not as a definition of the limits of the invention.

FIG. 1 shows the DNA profile of p53-positive and p53-compromised cells after treatment with VX-680. (a) p53-positive cells (A375, U2OS, A549 and HCT116 p53+/+) and (b) p53-compromised cells (HCT116 p53−/−, MDA-MB-486 and H1299) were treated with 200 nM VX-680 and harvested at the indicated time points. Analysis of cell cycle distribution using propidium iodide staining was performed using flow cytometry. Representative DNA profiles are shown. DNA content is indicated. No DNA profile was shown for cells that are 100% apoptotic. The results show that inhibition of aurora kinase induces endoreduplication and apoptosis in tumour-derived cell lines expressing wild-type p53.

FIG. 2 shows that VX-680 induces tetraploid G2 arrest.

(a) shows the cell cycle distribution of A549 cells incubated with 200 nM VX-680 assessed at the specified time points, over a 48 hour time course.

(b) is a series of western blots showing the kinetics of Kinetics of Cdk/cyclin kinase activities after incubation with VX-680. Cdk/cyclin kinase activities were assayed in vitro (refer to Materials and Methods in Examples). Cdk2, cyclins A and B1 were immunoprecipitated from cell lysates using specific antibodies followed by in vitro kinase assays using histone H1 as a substrate. Suc1 is used to capture Cdk1 and Cdk2 complexes by protein pull down as described in Materials and Methods.

(c) is a series of western blots showing the comparison of expression levels of cell cycle-related proteins (Rb, cdc2, Cdk2, cyclin A2, cyclin E1 and cyclin B1) and p53-dependent gene products (MDM2 and p21) at the indicated time points after VX-680 treatment. Downregulation of mitotic cyclins A and B and upregulation of p53-dependent genes p21 and MDM2 correlate with the activation of p53.

(d) is schematic illustration showing the quantification of the mRNA levels of cyclins A and B, p21 and MDM2 in A549 cells incubated with VX-680 by qRT-PCR.

(e) is a comparison of the expression levels of cell cycle-related proteins in p53+/+ cells vs p53−/− cells. HCT116 p53−/− and p53−/− cells were treated as described in (a). Protein lysates were analyzed by western blot for the indicated proteins.

FIG. 3 shows the Effects of p53 or p21 deletion on the levels of p53 transcription targets in HCT116 cells treated with VX-680. mRNA levels of p53 transcriptional targets were assessed at specific time points after exposure of HCT116 cells to VX-680 over a time course of 48 h. Transcript levels of p53 transcriptional targets, p21, MDM2 and PUMA were significantly decreased in p53−/− and p21−/− cells when compared with the wild-type parental cells. It is noteworthy that cyclins A2 and B1 levels remained high in p53−/− and p21−/− cells even after cells were incubated with VX-680 for 48 h, in contrast to that observed in the wild-type cells.

FIG. 4 shows that ectopic expression of p53 and p21 rescues cells from endoreduplication.

(a) is a schematic illustration showing the DNA profiles in VX-680 treated A549 cells compared with A549 p53 knockdown cells. Ectopic expression of p53 and p21 rescues cells from endoreduplication. A549 cells were transduced with recombinant lentiviruses carrying the siRNAresistant p53 gene, p53siRes, which contains four silent mutations in siRNA binding sequence. Stable cell line expressing p53siRes (A549p53siRes) was transfected with p53-specific siRNA before incubating with VX-680 for another 72 h. DNA profiles were analyzed by FACS and protein lysates were prepared and analyzed by western blotting for the indicated proteins. A549 transduced with empty vector was used as control.

(b) Overexpression of p21 in HCT116 p53−/− was achieved using recombinant adenovirus carrying CMV-promoter driven p21 (Ad-CMVp21). At 8 h after transduction, cells were incubated with VX-680 for 72 h and cell cycle profile was analyzed by FACS. Polyploidy (48N) was represented as a percentage of total cell population. Results are representative of three independent experiments. MOI, multiplicity of infection; C, control

FIG. 5 shows that Nutlin-3 suppressed endoreduplication in cells with functional p53.

(a) shows the results of BrdU assays using A549 cells. Cells were treated with 200 nM VX-680 for 24 h and incubated with 10 mM BrdU for 30 min. Control cells and cells treated with nutlin-3 only or nutlin-3 in combination with VX-680 were assessed for BrdU incorporation which reveals endoreduplicating cells after VX-680 treatment. BrdU-positive cells in untreated control sample (upper left panel) and BrdU-positive cells that are 44N DNA (endoreduplicating cells) (upper right panel) are highlighted in the boxes and the percentages over the total cell population are indicated.

(b) is a schematic diagram showing the timing and order of addition of drugs. At 48 h after VX-680 treatment, cells were washed in drug-free media and incubated in drug-free media for another 48, 96 or 120 h. Cell cycle distribution was analyzed by flow cytometry. Also shown are the DNA profiles obtained using the drug treatment protocol depicted in (a). Cells pretreated with nutlin-3 assume a normal cell cycle profile comparable to the untreated control cells after recovery from drug treatment.

(c) is a schematic illustration of the DNA profiles obtained when human keratinocytes were treated with the indicated concentrations of either VX-680 or nutlin-3 and were harvested for cell cycle analysis at 48 h after drug treatments.

(d) shows the DNA profiles obtained when keratinocytes were either incubated with VX-680 (200 nM) or nutlin-3 (5 mM) for 64 h or pretreated with nutlin-3 (5 mM) for 16 h followed by incubation with VX-680 (200 nM) or nutlin-3 (5 mM) for a further 48 h. Also shown are bright field images of keratinoctyes at the end of the incubation period under the various indicated drug conditions are shown. Arrowheads indicate endoreduplicated cells. The results indicate that Nutlin-3 protects primary human keratinocytes from endoreduplication with minimal toxicity.

FIG. 6 shows that wild-type p53 is required for nutlin protection.

(a) is a comparison of the DNA profiles of P53+/+ and p53−/− cells after treatment with VX-680. HCT116 p53+/+ and p53−/− cells were incubated with 200 nM VX-680 and cells were harvested at the indicated time points for analysis of cell cycle distribution. Increased endoreduplication was observed in HCT116 p53−/− at later time points (40 and 48 h).

(b) is a comparison of the DNA profiles of p53+/+ and p53−/− cells with pre-treatment of Nutlin before incubation with VX-680. The results indicate that pretreatment protects HCT116p53+/+ but not p53−/− cells from VX-680-induced endoreduplication. Cell cycle analysis was performed comparing HCT116 p53+/+ and p53−/− cells that were pretreated with nutlin-3 (5 mM) for 16 h, followed by VX-680 (200 nM) and nutlin-3 (5 mM) for another 48 h.

(c) shows the results of colony formation assays used to assess the proliferative potential of A549 and HCT116 cells after nutlin-3 and VX-680 treatment. Cells were plated in 10-cm plates and exposed to nutlin-3 or DMSO (0.1%) for 16 h before incubation with VX-680 for 48 h. Colonies were counted 2 weeks after recovery in drug-free media. Cells were fixed with glutaraldehyde and stained with crystal violet. Plates are scanned and colonies were counted with an automated image analyzer. Three independent experiments were performed for each cell line.

(d) shows graphs which indicate the results of the colony formation assays. Each data point represents the mean±S.D.

(e) is a schematic illustration of fluorescent microscopy images of HCT116 p53+/+ and p53−/− cells that were transduced with recombinant lentiviruses carrying GFP and RFP genes, respectively. The cells were selected in zeocin for 2 weeks to achieve the stable cell lines, HCT116 p53+/+GFP and HCT116 p53−/− RFP. Equal numbers of HCT116 p53+/+GFP and p53−/− RFP were mixed and seeded to achieve 30% growth confluency. Cells were pretreated with nutlin-3 (5 mM) for 16 h before incubation with the indicated concentrations of VX-680 for another 48 h. Cells were then washed twice with drug-free media and allowed to recover for 5 days in fresh complete media. Microscopy images were taken with Deltavision (Axio). Nutlin-3 and DMSO (0.1%) treated controls are shown. Insets 1 and 2 show higher magnification of the images on the left.

FIG. 7 shows that Nutlin-3 protects cells from apoptosis induced by VX-680.

(a) Upper panel: Graphs show the quantification of the percentages of annexin V-positive cells in the p53-positive cells (A375, HT116p53P/p) and p53-compromised cells (HCT116p53_/_, MDA-MB-486) treated using a similar drug dosage regimen described in FIG. 6b. Light grey columns represent nutlin pretreatment pVX-680 and dark grey columns represent VX-680 only treatment. Results are representative of three independent experiments. Lower panel: A375 cells were pretreated with 5 mM of nutlin-3 (or with DMSO as control) for 16 h before incubation with VX-680 for 48 h. Cells were then harvested and stained for annexin V to detect apoptotic cells.

(b) shows that the reversal of the order of drug addition results in synergistic activation of apoptosis. Annexin V-positive fractions were indicated as percentages in the boxes. Conditions for drug treatment on A375 cells: (1) control; (2) nutlin-3; (3) pretreatment with nutlin-3 followed by 48 h incubation with VX-680pnutlin-3; (4) VX-680 and (5) pretreatment with VX-680 followed by 48 h incubation with VX-680pnutlin-3; and (6) combined addition of nutlin-3 and VX-680. Nutlin-3 was used at 5 mM and VX-680 at 200 nM for all indicated drug conditions.

(c) shows the western blot analysis of the A375 cells treated as described in b. The numbers 1-6 refer to the different conditions of treatment as described above in b. Bc1-2, p53 and p53-responsive gene products p21, MDM2 and Bax were detected using antibodies. Representative blots are shown. Each column on the right represents the ratio of intensity of Bax/Bc12 bands, which is calculated by dividing the densitometric value of Bax by the corresponding Bc12 value.

(d) is a schematic illustration showing the comparison of the effects of VX-680, Nutlin-3pVX-680 and the reverse drug combination, VX-680 p Nutlin-3, on the survival of A375 cells. Colony survival assay was performed as described in FIG. 6c. Each data point represents the mean±S.D. (n=3)

FIG. 8 shows the accumulation of p53 and p53-dependent gene products, p21, MDM2 and PUMA in response to increasing concentrations of VX-680.

(A) Western blots show the accumulation of p53, p21, MDM2 and PUMA proteins. Graphs below represent the quantification of the corresponding transcript levels of the proteins.

(B) is a graph showing the Mitotic index of A549 cells exposed to VX-680. A549 cells were incubated with VX-680 (200 nM) and the mitotic cells were counted and expressed as a percentage of total cells count. To aid visualization of the condensed chromosomes, H2B-GFP was expressed in A549. Each data point represents the mean±SD (n=3).

FIG. 9 shows that Nutlin-3 suppressed endoreduplication in response to VX-680 in A549 cells but not in A549 cells transfected with p53 siRNA.

(A) A549 cells pretreated with nutlin-3 (5 μM) followed by VX-680 (200 nM) remained arrested at G1, in contrast to cells that were treated with VX-680 only.

(B) Extent of endoreduplication is exacerbated in cells with compromised p53 functions. A549 cells transfected with p53 siRNA (Dharmacon pool) were incubated with VX-680 (200 nM) for the indicated time periods after transfection. In a parallel experiment, p53 siRNA transfected cells were pretreated with nutlin-3 (5 μM) before incubating with VX-680 for 72 h. DNA-dependent cell cycle analysis was performed using flow cytometry.

(C) Efficiency of knockdown of p53 is established by comparing the protein expression levels of p53 and p53-transcription dependent gene products, p21 and MDM2, in mock-transfected and p53 siRNA transfected cells.

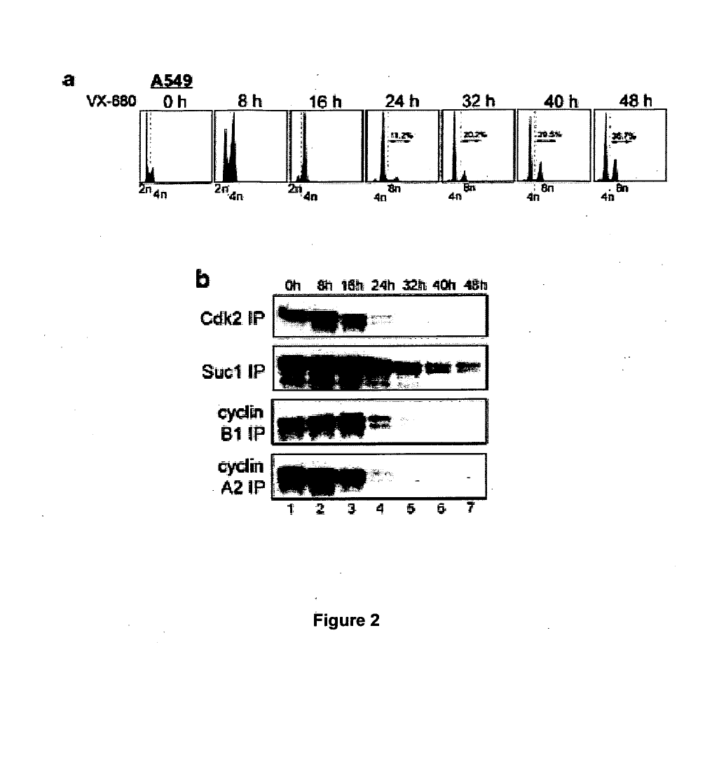

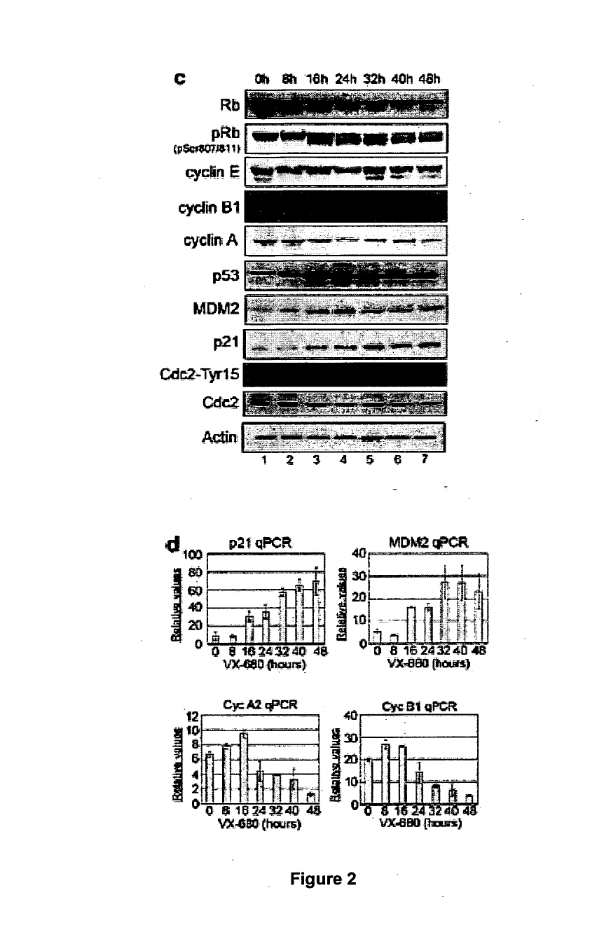

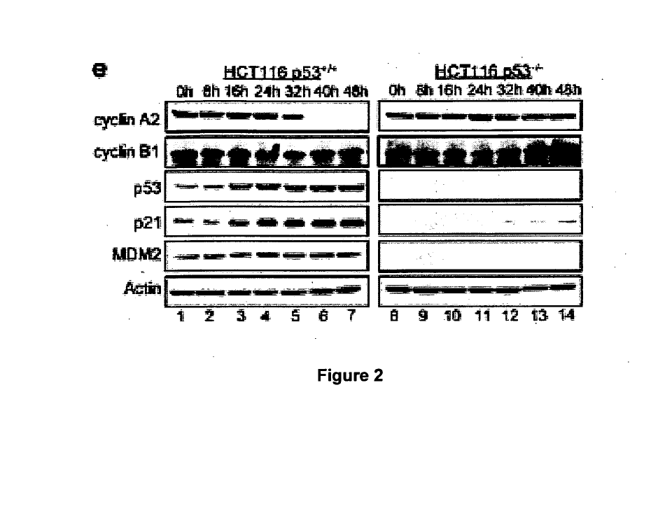

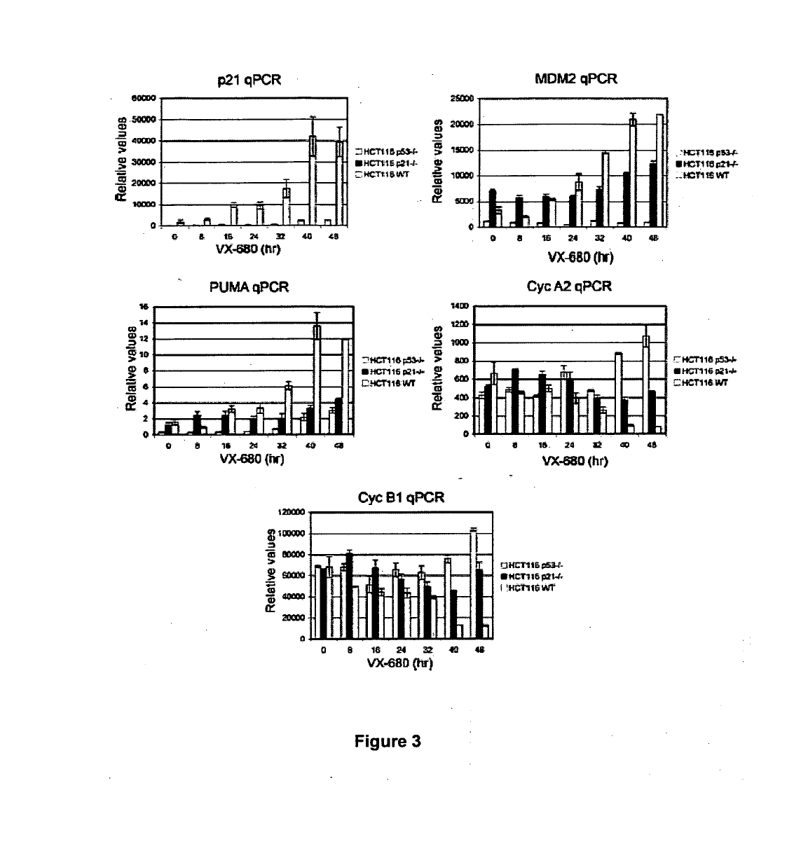

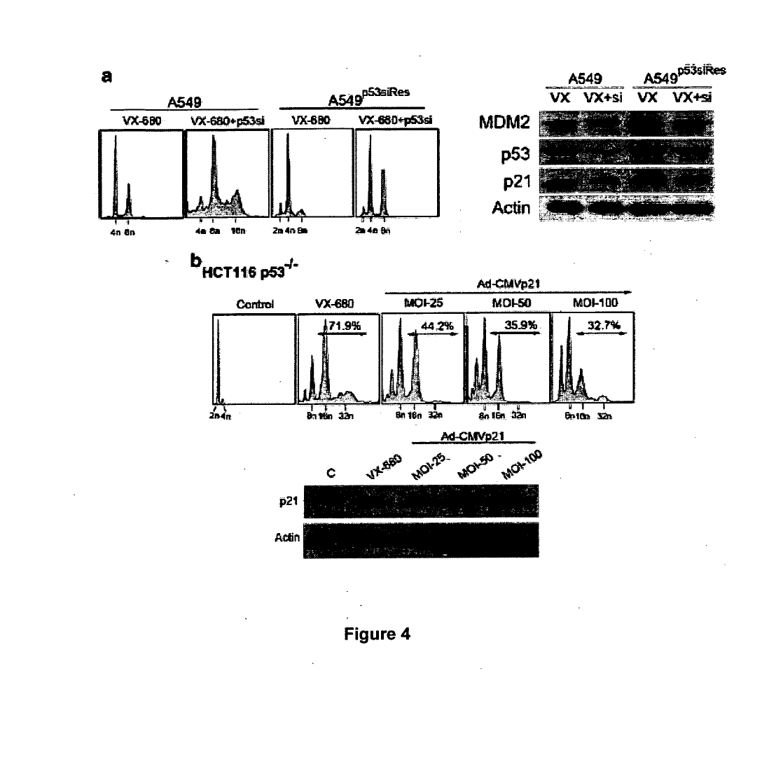

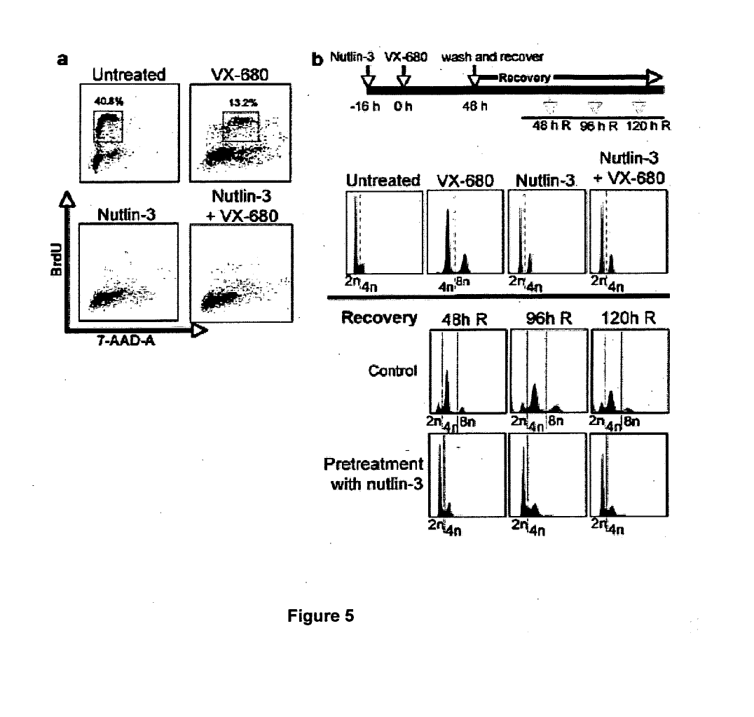

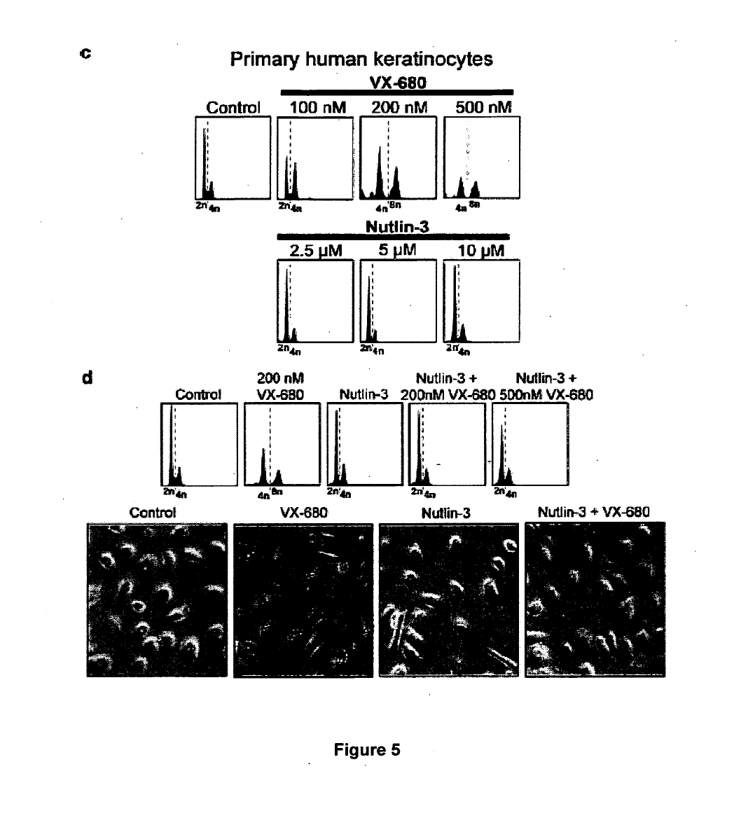

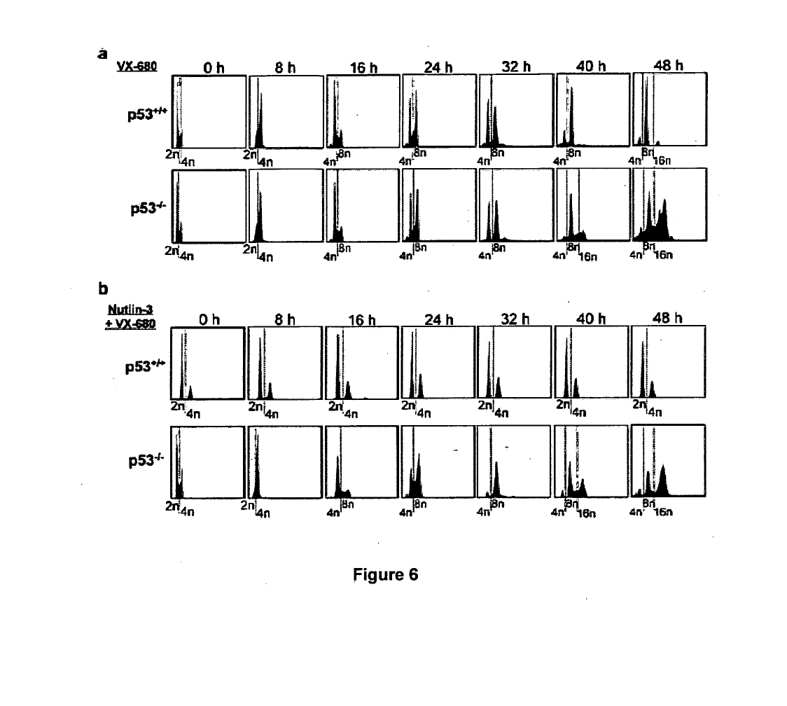

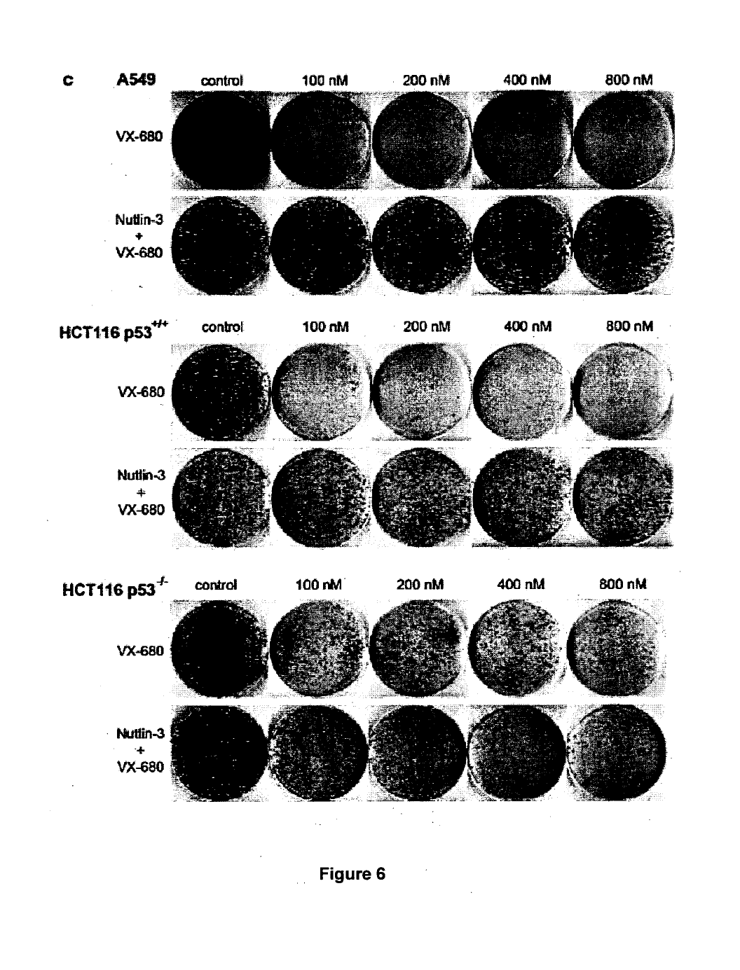

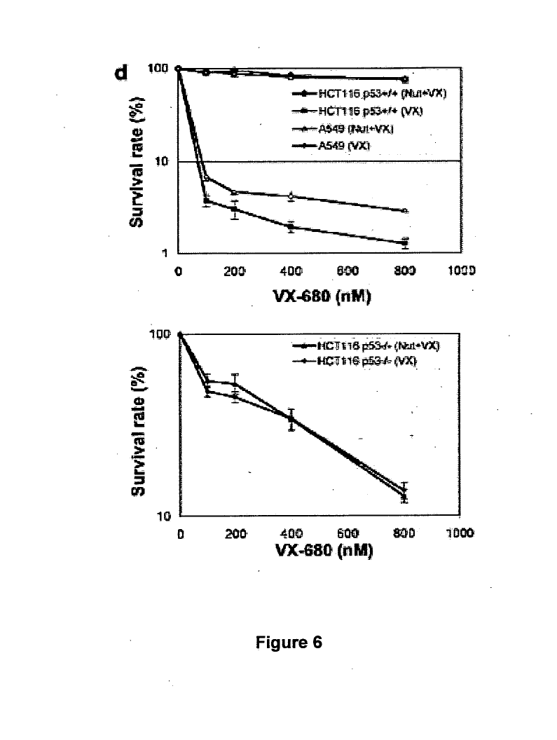

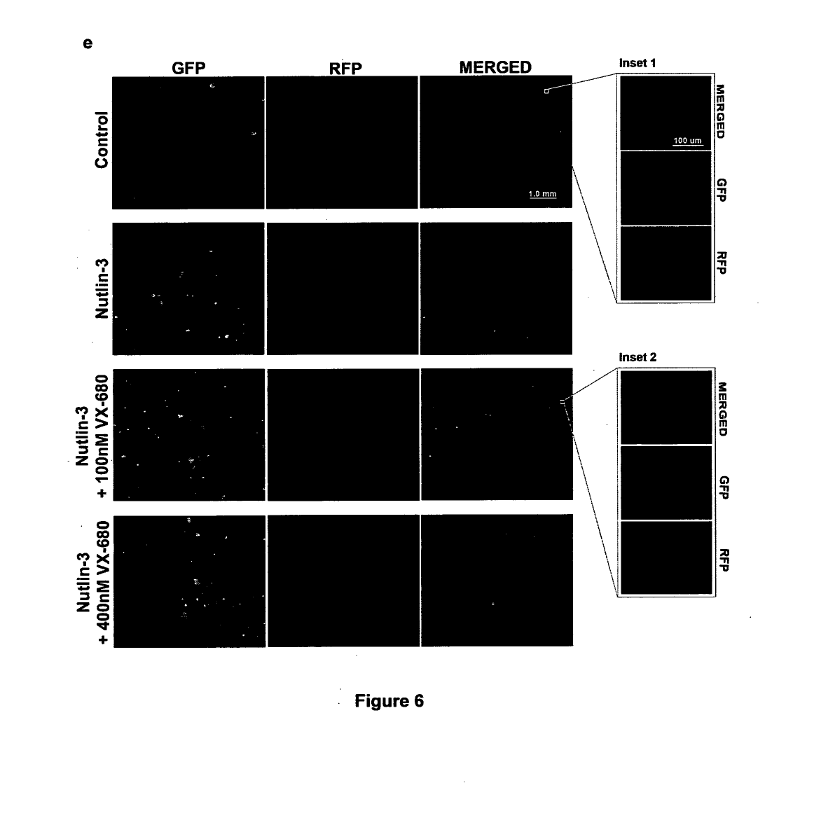

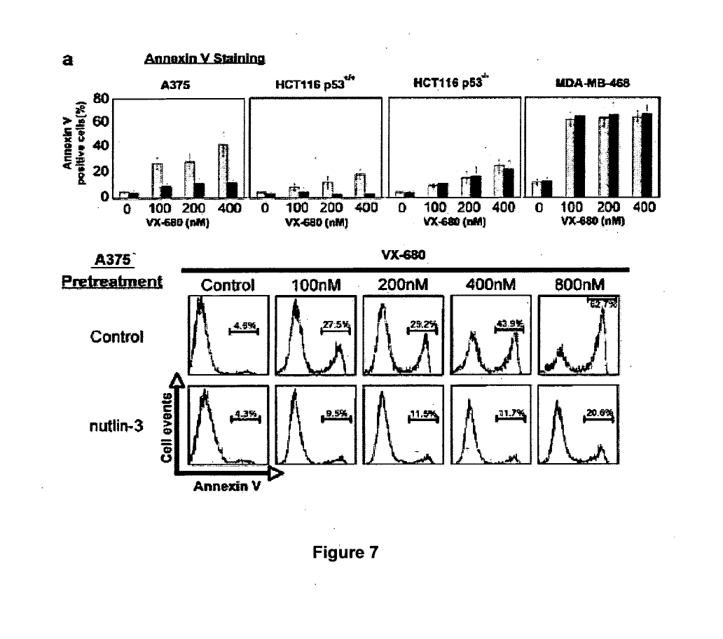

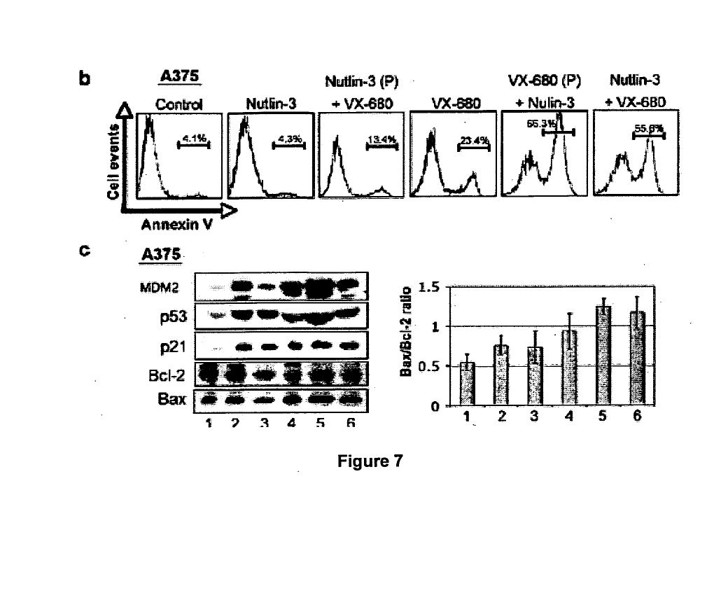

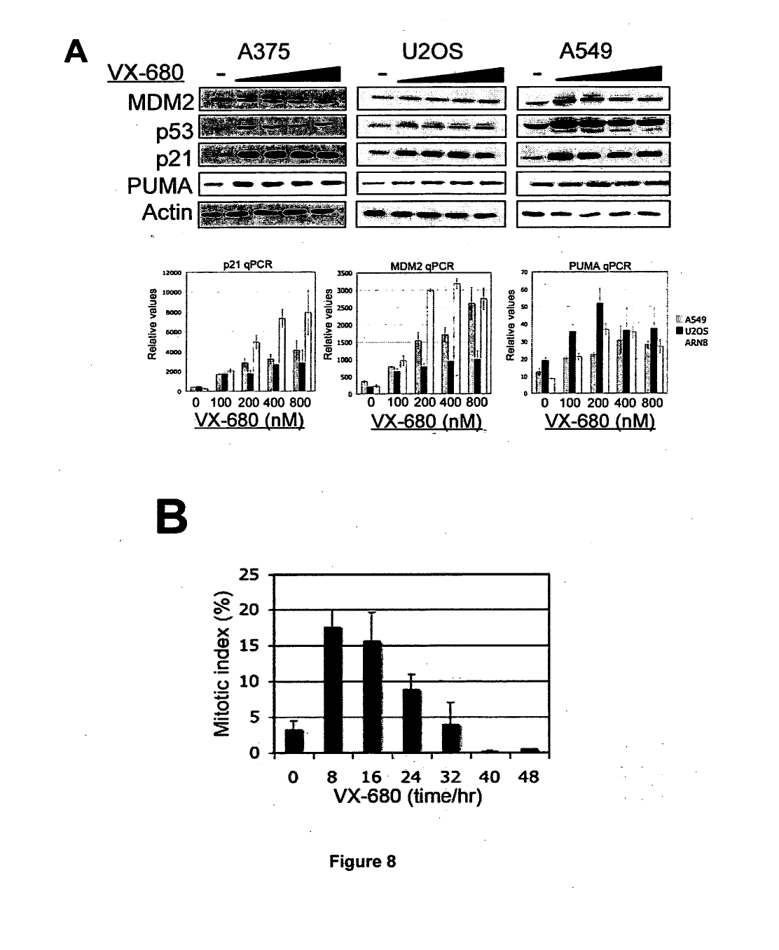

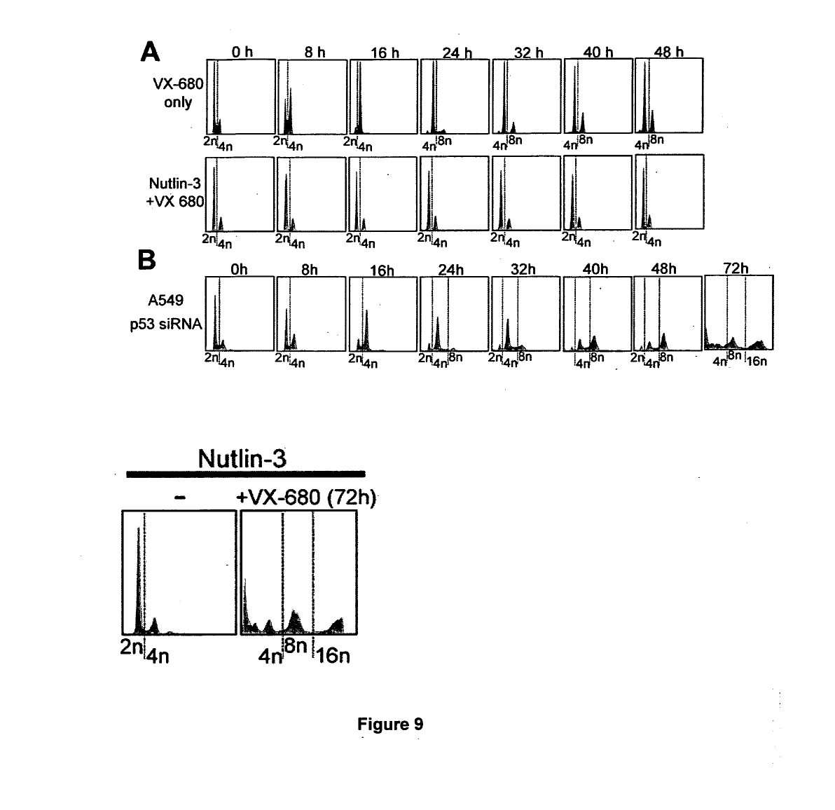

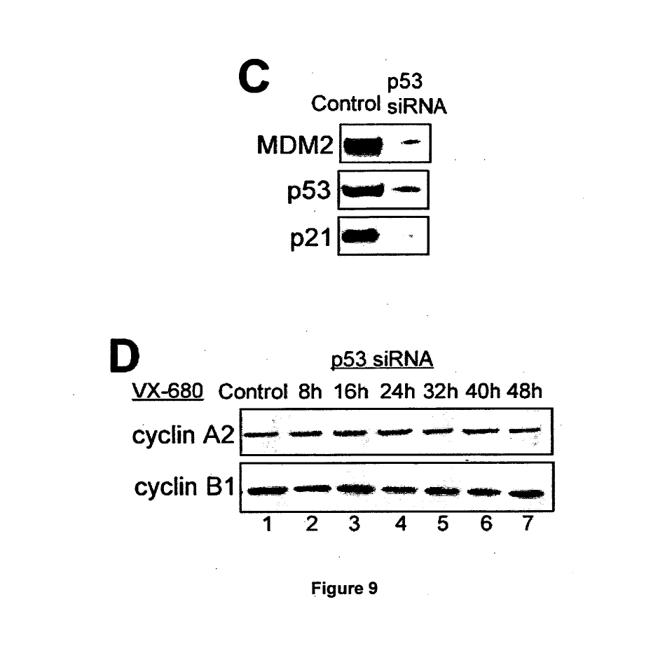

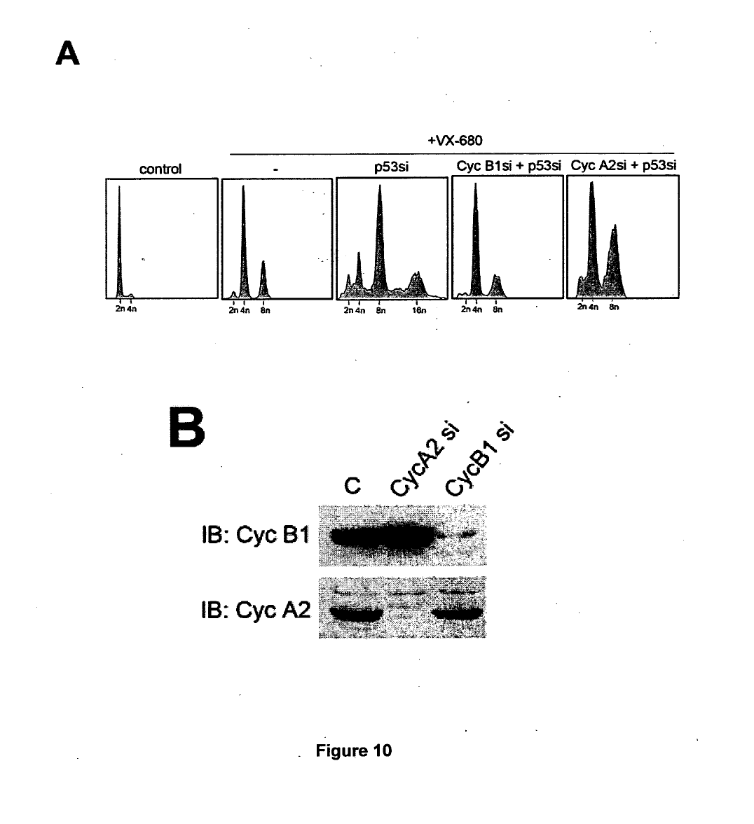

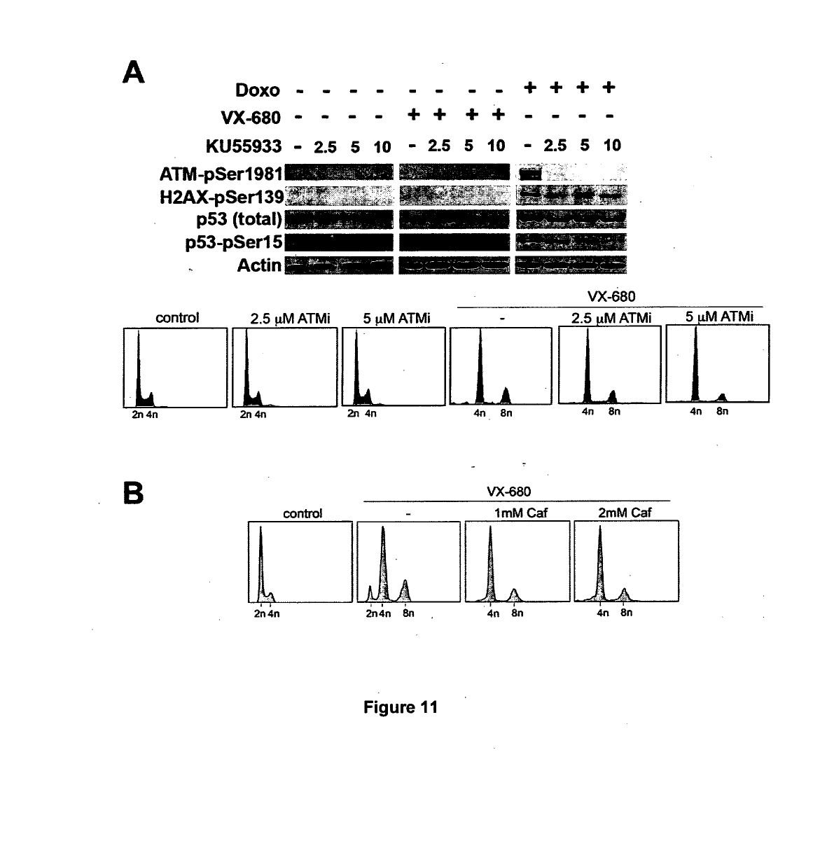

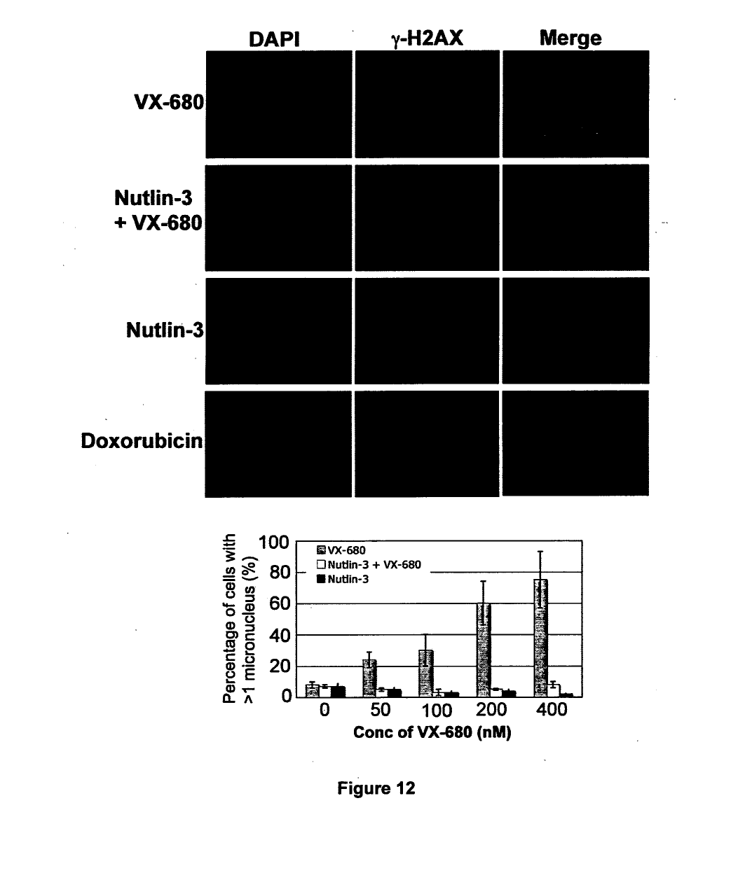

(D) Representative blots showing the accumulation of cyclins A and B in the presence of VX-680 in p53 siRNA treated A549 cells, in contrast to the untransfected cells shown in FIG. 2A.

FIG. 10 shows that the Downregulation of cyclin A2 and cyclin B1 suppressed endoreduplication.

(A) is a series of representative DNA histograms showing the results of A549 cells transfected with siRNA to p53 only or in combination with either siRNA to cyclin A2 or cyclin B1. Transfected cells were incubated with VX-680 for 72 hours before being harvested for DNA analysis using FACS. Control cells were mock-transfected and incubated with 0.1% DMSO.

(B) is a series of western blots which confirm the downregulation of cyclin A2 and cyclin B1 by gene-specific siRNAs. Protein lysates were prepared from cells harvested 48 hours after transfection.

FIG. 11 shows that VX-680 induces a caffeine-resistant G2 arrest.

(A) Western blots show the accumulation of phospho-S1981 ATM, phospho-H2AX, phospho-S15 p53 in response to 1 μM doxorubicin but not to VX-680. ATM inhibitor (KU55933) abrogates the observed phosphorylation in a dose-dependent manner. DNA histograms (lower panel) show the induction of G2 arrested cells with 8n DNA content after 48 hours of drug treatment with VX-680 and KU55′933 (ATMi) or caffeine as in (B).

FIG. 12 shows that VX-680 induces genomic instability and micronuclei formation. A549 cells were incubated with 1) VX-680 only for 48 hours 2) with nutlin-3 for 16 hours before adding VX-680 for another 48 hours 3) nutlin-3 only for 48 hours or 4) doxorubicin for 16 hours. Cells were fixed and stained for γH2AX (green) as described in Materials and Methods in the Examples. Nuclei were counterstained with DAPI blue. Visualization of nuclei and γH2AX positive foci was done by fluorescence microscopy. Graph below shows the quantification of micronuclei (based on 200 nuclei) per drug treatment condition.

### TABLES

Table 1 shows the tumour-derived and primary cell lines used.

Table 2 shows the primers and their sequences used for the cloning of lentiviral vectors.

### SEQUENCES

SEQ ID NO:1—Forward primer used for amplifying p53 cDNA to produce expression clone pLenti4-p53. Highlighted in bold is the attB1 site.
SEQ ID NO:2—Reverse primer used for amplifying p53 cDNA to produce expression clone pLenti4-p53. Highlighted in bold is the attB2 site.
SEQ ID NO:3—Forward primer used for amplifying GFP cDNA to produce expression clone pLenti4-GFP. Highlighted in bold is the attB1 site.
SEQ ID NO:4—Reverse primer used for amplifying GFP cDNA to produce expression clone pLenti4-GFP. Highlighted in bold is the attB2 site.
SEQ ID NO:5—Forward primer used for amplifying GFP cDNA to produce expression clone pLenti4-RFP. Highlighted in bold is the attB1 site.
SEQ ID NO:6—Reverse primer used for amplifying GFP cDNA to produce expression clone pLenti4-GFP. Highlighted in bold is the attB2 site.
SEQ ID NO:7—Forward primer used for incorporating base changes into pLenti4-p53 plasmid in order to produce pLenti4-p53siRes construct.
SEQ ID NO:8—Reverse primer used for incorporating base changes into pLenti4-p53 plasmid in order to produce pLenti4-p53siRes construct.
SEQ ID NO:9—siRNA oligo for knockdown of p53 expression.
SEQ ID NO:10—siRNA oligo for knockdown of cyclin A2 expression.
SEQ ID NO:11—siRNA oligo for knockdown of cyclin B1 expression.

### DETAILED DESCRIPTION

### Examples

Non-limiting examples of the invention, including the best mode, and a comparative example will be further described in greater detail by reference to specific Examples, which should not be construed as in any way limiting the scope of the invention.

### Materials and Methods

### Cell Lines, Primary Lines and Culture Conditions

A549, U2OS, A375, H1299 and MDA-MB-486 were purchased from ATCC (Manassas, Va., USA) and cultured in DMEM (Invitrogen, Carlsbad, Calif., USA) supplemented with 1% penicillin/streptomycin (Invitrogen), 10% FBS and 2 mM glutamine (GIBCO, Carlsbad, Calif., USA). Wild-type HCT116, HCT116p53−/− and HCT116p21−/− cells were kind gifts from Dr B Vogelstein (John Hopkins University School of Medicine, Baltimore, Md., USA). HCT116 and derivatives were cultured in McCoy's 5A media (Sigma, St Louis, Mo., USA) supplemented with 1% penicillin/streptomycin, 10% FBS and 2 mM glutamine. Primary human keratinocytes were purchased from Invitrogen and cultured in defined keratinocyte serum-free media (Invitrogen). All cell lines were cultured in a CO₂ incubator (5% CO2 and 21% O2) at 37° C.

TABLE 1
Tumor derived and primary cell lines used

| Cell line | p53 status |
|---|---|
| A375 melanoma | + |
| A549 lung adenocarcinoma epithelial | + |
| U2OS osteosarcoma | + |
| HCT116 colorectal carcinoma | + |
| HCT116p53^{-/-} colorectal carcinoma | − |
| H1299 lung carcinoma | − |
| MDA-MB-468 breast adenocarcinoma | R273H mutation |
| Human primary keratinocytes | + |

### Antibodies and Reagents

Antibodies against p53 (DO-1), p21 (Ab118) and MDM2 (2A9) were kindly provided by Dr B Vojtesek (Masaryk Memorial Cancer Institute, Bruno, Czech Republic). Antibodies against cyclin E (M−20), cyclin B1 (GNS1), cyclin A2 (H432) and Rb (Rb1) were from Santa Cruz Biotechnology (Santa Cruz, Calif., USA). Phospho-Rb (Ser807/811), phospho-ATM (Ser1981; 10H11.E12) and Phospho-cdc2 (Tyr15; 10A11) were from Cell Signaling (Danvers, Mass., USA). V5 epitope antibody was from Invitrogen. Antibody for IP of Cdk2 was described previously.¹² Suc1 conjugated to agarose beads was from Millipore Upstate (Billerica, Mass., USA) (14-132.) Nutlin-3 was from Calbiochem (San Diego, Calif., USA) and VX-680 was from American Customs Chemical Corporation (San Diego, Calif., USA). Both compounds were reconstituted in DMSO. For use in cell treatment, the final DMSO concentration in the media did not exceed 0.1% (v/v).

### Plasmids and Primers

Cloning of lentiviral vectors: cDNA of the respective genes was amplified by PCR using the following gene-specific primers:

TABLE 2
Gene-specific Primers

| | SEQ | |
|---|---|---|
| Primer | ID | |
| Name | NO | Sequence (5′ - 3′) |
| p53 (F) | 1 | GGGGACAAGTTTGTACAAAAAAGCAGGCTTCGAAGGAG |
| | | ATAGAACCATGGAGGAGCCGCAGTCAGA |
| p53 (R) | 2 | GGGGACCACTTTGTACAAGAAAGCTGGGTCTCAGTCTG |
| | | AGTCAGGCCCTTC |
| GFP (F) | 3 | GGGGACAAGTTTGTACAAAAAAGCAGGCTTCGAAGGAG |
| | | ATAGAACCATGGTGAGCAAGGGCGAGGAGC |
| GFP (R) | 4 | GGGGACCACTTTGTACAAGAAAGCTGGGTCTTACTTGT |
| | | ACAGCTCGTCCATG |
| RFP (F) | 5 | GGGGACAAGTTTGTACAAAAAAGCAGGCTTCGAAGGAG |
| | | ATAGAACC GCCTCCTCCGAGGACGTCATC |
| RFP (R) | 6 | GGGGACCACTTTGTACAAGAAAGCTGGGTCCTAGGTCT |
| | | CGATCGAGGTCGAC |

pBI-p73 wild-type/EGFP,¹³ (Dr B Vogelstein, Addgene) was used as the template for PCR. Highlighted in bold are the attB1 and attB2 sites. PCR fragments are recombined with the pDONR vector (Invitrogen) using BP clonase according to the manufacturer's protocol. Successful clones were verified by sequencing.

Expression clones pLenti4-p53, pLenti4-GFP and pLenti4-RFP were generated through recombination of the verified pDONR vectors and pLenti4-V5-DEST vector (Invitrogen) using LR clonase (Invitrogen). Successful clones were verified by sequencing. The pLenti4-p53 plasmid was used as a template for the generation of the pLenti4-p53siRes construct. Primers (fwd) 5′-GAGTGGAAGGA AGTTCGCATGCGGAGTATTTGGATGACAG-3′ (SEQ ID NO:7) and (rev) 5′-CTGTCATCCAAATA CTCCGCATGCGAACTTCCTTCCACTC-3′ (SEQ ID NO: 8) were used to incorporate the base changes (underlined) using QUIKChange site-directed mutagensis kit (Stratagene, La Jolla, Calif., USA).

siRNA Transfection

p53 siRNA oligo 1 (p53si) 5′-GCAGUCAGAUCCUA GCGUCUU-3′ (SEQ ID NO: 9), cyclin A2 siRNA 5′-CTUCUTTGUTUGGTTCCTG-3′ (SEQ ID NO: 10) and cyclin B1 siRNA 5′-UCTTTCGCCTGUGCCTUTT-3′ were from Dharmacon (Lafayette, Colo., USA) (SEQ ID NO:11). Cells were plated in six-well dishes overnight in antibiotic-free media. Cells were incubated with Optimen (GIBCO) for an hour before transfection using Lipofectamine 2000 (Invitrogen) and siRNA (30 nM) according to the manufacturer's recommendation. Cells were either harvested at 48 h after transfection or incubated with VX-680 for another 48 h before analysis.

### Viral Production and Transduction

293T cells were transfected with pLenti4-empty vector or pLenti4 construct carrying the gene of interest using Lipofectamine 2000 (Invitrogen). Supernatants were collected at 48 and 72 h after transfection, filtered through a 0.45 mM filter and concentrated through ultracentrifugation. Viral titers were estimated using serial dilutions of the concentrated virus stock and determining the number of antibiotic (zeocin) resistance colonies at 3 weeks after transduction and selection. On the average, viral titers were estimated to be 5.6×10⁷ to 2×10⁸ TU/ml.

In vitro transduction was performed by plating cells in 24-well plates in DMEM supplemented with 10% FCS. After overnight incubation, cells were transduced with an appropriate titer (5 MOI) and incubated overnight. The next day, fresh media was added and incubated for another 24 h before cells were selected for antibiotic resistance in zeocin (250 μg/ml) containing media for 2 weeks. Ad-CMVp21 was purchased from Vector Biolabs (Philadelphia, Pa., USA). HCT116p53−/− cells were incubated in McCoy's media (without serum) containing the adenovirus for an hour. The virus-containing media was removed and replaced with fresh media containing 200 nM of VX-680. Cell cycle analysis was performed using fluorescence-activated cell sorting. (FACS) at the end of 72 h of drug treatment. The results were analyzed using FlowJo software (TreeStar Inc., Ashland, Oreg., USA) and percentages of polyploidy cells (count of polyploidy cells over the total parent cell population) were calculated.

### FACS and Apoptosis Analysis

For analysis of cell cycle distribution, cells were harvested and fixed in 70% ethanol/PBS solution. Cells were stained in propidium-iodide containing solution (25 μg/ml propidium iodide supplemented with 1 mg/ml RNase A, in PBS (pH 7.8); Sigma Chemical, St Louis, Mo., USA) for 15 min at room temperature.

For analysis of apoptosis, cells were harvested without fixation. Apoptosis was evaluated using the annexin V (fluorescein isothiocyanate (FITC))-propidium iodide binding assay (Roche, Indianapolis, Ind., USA). The extent of apoptosis was quantified as a percentage of annexin V-positive cells over the total cell population. Flow cytometric analysis was performed on a BD LSR II System (BD Biosciences, Stockholm, Sweden). Data were analyzed using FlowJo software and ModFit LT (Verity Software House, Topsham, Me., USA).

### Colony Survival Assay

A total of 1000 cells were plated on each 10 cm plate and allowed to adhere overnight. Cells were either left in media with 0.1% DMSO as control or incubated with 5 mM nutlin-3 for 16 h before adding the indicated concentrations of VX-680. After 48 h, cells were washed and recovered in fresh media for 12 days. Cells were then fixed in 6% glutaraldehyde (Sigma) and stained with crystal violet. Colonies were counted using GelCount™ (Oxford Optronix, Oxford, England). Each condition was carried out in triplicate.

### Western Blot and Immunoprecipitation

For western blotting, cell extracts were prepared using NP-40 lysis buffer (50 mM Tris-HCl (pH 8.0), 150 mM NaCl, 1% NP-40, 5 mM EDTA supplemented with protease and phosphatase inhibitor cocktails (Sigma).

For immunoprecipitation, 0.1×10⁷ cells were harvested and lysed in extraction buffer (50 mM HEPES, pH 7.4, 150 mM NaCl, 10 mM b-glycerophosphate, 10% glycerol, 0.5% Tween-20, 1.0 mM EDTA, 2.5 mM EGTA, 0.5 mM DTT and protease inhibitors (10 μg/ml each of leupeptin, chymostatin and pepstatin (Chemicon, Temecula, Calif., USA)). Cells were incubated with the extraction buffer for 30 min at 41 C followed by centrifugation at 14 000 r.p.m. at 41 C for 30 min. A total of 3-5 μg of antibody were incubated with lysates (300 μg) in 750 μl of EBN buffer (80 mM b-glycerophosphate, pH 7.3, 20 mM EGTA, 15 mM MgCl2 and 0.5% NP-40) containing 1 mg/ml ovalbumin, 2 mM NaF and protease inhibitors for 3 h at 41 C. Then, 10 μl of protein G beads was added to the mix and incubated for another hour. Protein immunoprecipitates were washed twice in EBN buffer and twice in EB buffer (EBN without the NaCl and NP-40).

### Kinase Assay

The immunoprecipitated beads were resuspended in 5 μl of EB buffer, 10 mM DTT and 20 to 50 mM μTP. Each sample was incubated with 5-10 μCi [γ-32P]ATP, 1.5 μg histone H1 (Roche, no. 1004875) in a final volume of 16 μl. After incubation for 30 min, reactions were terminated by the addition of 5 μl 5×SDS-PAGE sample buffer. After electrophoresis on 12.5% polyacrylamide gels, phosphorylation was analyzed by autoradiography and quantified by phosphorimage analysis.

### BrdU Labeling

Cells were incubated in DMEM containing 10 mM BrdU for 30 min in a 37° C./5% CO₂ incubator. Detection of BrdU-labeled cells was performed using the In Situ Cell Proliferation Kit from Roche. In brief, cells were harvested and fixed using a 70% ethanol/50 mM Glycine (pH 2.0) on ice. Cells were pelleted and resuspended in the HCl-denaturation solution at room temperature for 20 min. Cells were then pelleted and 50 μl anti-BrdU-FLUOS antibody was added to the cells and incubated for 45 min at 37° C. in a humidified chamber. For staining of total DNA, 7-AAD was added to the cells, followed by incubation at room temperature for 10 min. Cells were analyzed immediately using flow cytometry (BD LSR II System).

### Quantitative Reverse Transcription-PCR

Total RNA was isolated using the RNeasy kit (Qiagen, Valencia, Calif., USA). The RNA was quantified using spectrophotometric analysis and used for quantitative real-time PCR. The primers used for each target analyzed are available on request. The RNA Master Power SYBR Green Mix (Roche) was used for quantification of mRNA levels.

### Immunostaining and Fluorescence Microscopy

Indirect immunofluorescence was carried out as described previously.¹⁴ g-H2AX (Novus Biologicals, Littleton, Colo., USA, NB 100-383) and anti-rabbit Alexa 488-coupled secondary antibody (Molecular Probes, Carlsbad, Calif., USA) were used. Nuclei were counterstained with Hoechst 33342 (Molecular Probes). Immunofluorescence was visualized using Axiolmager Z1 (Zeiss, Gottingen, Germany).

### Results

### Example 1

### Aurora Kinase Inhibition Induces Extensive Endoreduplication in Both p53 Wild-Type and p53-Deficient Cell Lines

p53 has been implicated in a postmitotic G1 checkpoint in response to various mitotic inhibitors. We examined whether this checkpoint is induced after aurora kinase inhibition by VX-680 and whether tetraploidy is suppressed by wild-type p53 functions. Using a panel of commonly used tumor-derived cell lines expressing either wild-type p53, mutant p53 or a deletion at the p53 gene locus (Table 1), we tested the correlation of p53 gene status to VX-680-induced endoreduplication. These cell lines were treated with VX-680, and harvested for cell cycle analysis at 24, 48 and 72 h.

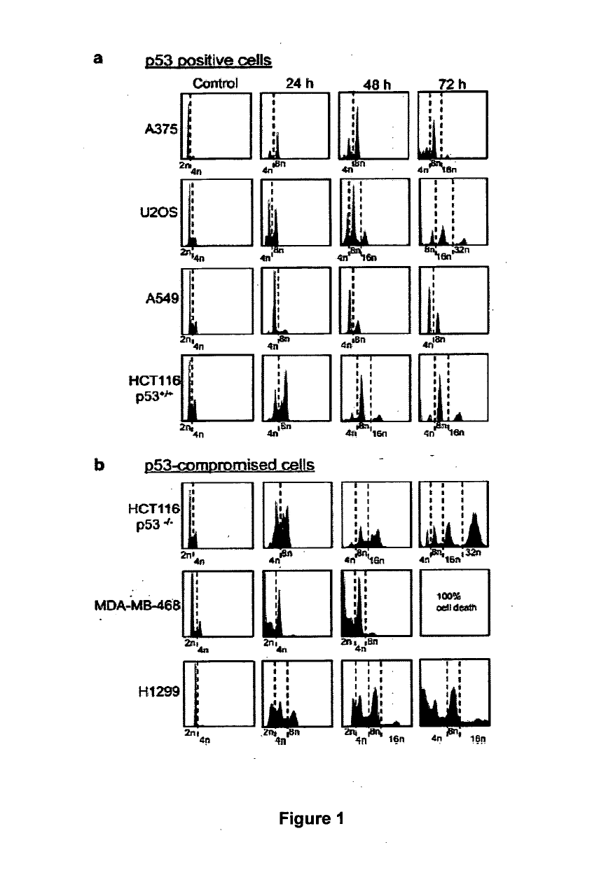

To our surprise, all tumor cell lines undergo endoreduplication. Despite the presence of wild-type p53 in A375, A549, U2OS and HCT116, endoreduplication occurs but to a variable extent, giving rise to cells with 8N, and, in some cases, 16N and 32N DNA content (FIG. 1). p53 was activated in cells treated with VX-680, and p53 target genes, p21, PUMA and MDM2 were all upregulated (FIG. 8A). Activation of p53 seemed to restrict further endoreduplication through imposing cell cycle arrest, as evident from the lack of S-phase cells at 48 and 72 h after drug treatment (FIG. 1). Taken together, our data suggest that p53 response is activated in response to aurora kinase inhibition but is insufficient to completely protect against endoreduplication.

### Example 2

### VX-680 Induces Tetraploid G2 Arrest

We did a careful time course analysis of the DNA content of A549 (p53 wild type) after VX-680 treatment and found that cells arrested briefly in G2/M at 16 h with predominantly 4N DNA content before a second round of DNA replication was initiated at 16 h (FIG. 2a). After 48 h, more than 35% of cells were arrested in tetraploid G2/M (8N) (FIG. 2a). Analysis of the mitotic indexes suggests that cells were arrested in G2 and not in mitosis; the mitotic index peaked at 16 h after treatment with VX-680 but subsequently decreased to 2% at 48 h (FIG. 8B). Together, the results confirmed that the 8N cells were arrested predominantly in G2 and VX-680 inhibited mitosis in part through a G2 checkpoint.

To elucidate the mechanism underlying the observed G2 arrest, we examined both cyclin A2-Cdk2 and cyclin B1-cdc2, protein complexes known to have crucial roles in the regulation of the G2-M transition.⁴⁻⁶ A careful time course analysis of the cyclin-associated kinase activities revealed a rapid reduction of cyclins A2- and B1-associated kinase activities (FIG. 2b). Cyclin-dependent kinase 2 (Cdk2) and cell division cycle 2 (cdc2) kinase activities were also markedly reduced to undetectable levels, but this was neither a result of substantial changes in the levels of cdc2/Cdk2 protein nor of inhibitory tyrosine phosphorylation.⁷ As the kinase activity of Cdk2 or cdc2 requires binding to the appropriate cyclins, we reasoned that the ablation of cdc2/Cdk2 kinase activities could be because of a change in the intracellular levels of cyclins A2 and B1. Indeed, a timedependent decrease in the protein levels of cyclins A2 and B1, correlating with the kinetics of suppression of Cdk activities (FIG. 2c), was observed.

### Example 3

### p53 Imposes a Caffeine-Resistant G2 Checkpoint in Part Through a p21-Dependent Repression of Cyclins A2 and B1 mRNAs

We noticed that both cyclins were depleted in A549 cells but remained unchanged in A549 cells transfected with p53 siRNA (FIG. 2c and FIG. 9D). Similarly, depletion of cyclin A2 protein and, to a lesser extent, cyclin B1 protein were observed in VX-680-treated wild-type HCT116 but not in the derivatives HCT116p53−/− or HCT116p21−/− (FIG. 2e; data not shown). Loss of cyclins A2 and B1 occurs at the transcriptional level as their mRNAs were greatly reduced in the wild-type cells but not in p53−/− or p21−/− cells (FIG. 3).

Given that the promoters of cyclins A2 and B1 genes contain p53 regulatory elements,⁸ a plausible explanation is that accumulated p53 protein directly represses the transcription of cyclins A2 and B1 genes. Alternatively, the p21 induced by p53 can bind directly to Cdk/cyclin complexes, resulting in the sequestration of E2F1 by hypophosphorylated retinoblastoma protein (pRb). The repression of cyclins A2 and B1 is specific, as cdc2 protein (a reported target of p53-mediated repression⁹), shows only a marginal decrease (FIG. 2c). Although it is tempting to speculate that p53 mediates the repression through a p21-dependent pathway, it is likely that p21-independent activity of p53 also contributes to the observed repression, as HCT116p53−/− cells show consistently higher levels of cyclins A2 and B1 transcripts when compared with HCT116p21−/− cells at later time points of VX-680 treatment (FIG. 3). Rescue of cyclins A2 and B1 expression coincided with increased polyploidy (FIGS. 3, 6a and FIG. 9). Conversely, siRNA-mediated downregulation of both genes, required for mitotic entry,^{5,10} contributes to the observed G2 arrest and prevents further endoreduplication in A549 transfected with p53siRNA (see Example 6 and FIG. 10). Ectopic expression of p53 or p21 suppressed polyploidy in p53-deficient cells (see Example 7 and FIG. 4).

Together, these data suggest p53 mediates the transcriptional repression of cyclins A2 and B1 genes, and depletion of cyclins A2 and B1 suppresses endoreduplication through a G2 checkpoint. Furthermore, caffeine, which inhibits ataxiatelangiectasia mutated (ATM) and ATR and effectively overrides the G2 checkpoint in response to DNA damage but not in response to cyclin A2 knockdown,¹⁵ did not override the G2 arrest induced by VX-680 (see Example 8 and FIG. 11).

### Example 4

### Previous Activation of p53-Dependent Cell Cycle Arrest Suppressed VX-680-Induced Endoreduplication and Apoptosis

We next asked whether we could activate p53 before VX-680 addition and prevent cells from transiting a failed mitosis leading to tetraploidy. Nutlin-3 has been shown to induce a p53-dependent arrest.^{1-3,11} Pretreating A549 cells with a low dose of nutlin-3 (5 mM) before VX-680 treatment arrested cells even after 48 h of VX-680 exposure. This is confirmed by a BrdU assay (FIG. 5a). Remarkably, nutlin-3-pretreated cells assume a normal diploid DNA profile after cells are recovered in drug-free media (FIG. 5b), in contrast to cells exposed only to VX-680.

We found that normal nontransformed human epithelial keratinocytes (HEKs) are also susceptible to tetraploidy formation as a result of aurora kinase inhibition. HEK cells treated with VX-680 underwent DNA replication without cell division, resulting in a significant tetraploid G2/M population (FIG. 5c). Nutlinpretreated HEK cells did not show significant tetraploidy and maintained high cellular viability even after 5 days in nutlin-3, therefore suggesting that normal cells can withstand prolonged exposure to nutlin-3 and activation of p53 (FIG. 5d and data not shown), consistent with other reports.^{1,3} Therefore, nutlin pretreatment suppresses ploidy in response to aurora kinase inhibition. In addition, nutlin also suppress apoptosis induced by VX-680 (see Example 9 and FIG. 7).

### Example 5

### Nutlin Confers a Long-Term Proliferative Advantage that Requires Wild-Type p53

Nutlin induced an arrest in HCT116p53+/+ but not in HCT116p53−/− (FIG. 6b). The p53 dependency of nutlin is also demonstrated in A549 with attenuated p53 (FIG. 9). The proliferative capacity of nutlin-pretreated cells was compared with cells treated only with VX-680 in a colony formation assay. VX-680 treatment alone drastically reduced the number of A549 colonies (FIG. 6c; upper panel). It is noteworthy that nutlin alone moderately decreases the number of surviving colonies. However, when the cells were pretreated with nutlin-3, the fraction of surviving colonies was enhanced up to 20-fold (FIGS. 6c and d). Similarly, HCT116p53+/+ cells show markedly better survival (up to 15-fold) when pretreated with nutlin-3 than in the presence of VX-680 alone. In contrast, nutlin-3 pretreatment did not affect the colony survival rate in p53-deficient HCT116 (FIGS. 6c and d).

We further studied the responses of p53 wild-type and p53-deficient cells in co-culture system, in which both cell types were cultured together for the duration of the experiment. HCT116p53+/+ and the HCT116p53−/− cells were distinguished using green fluorescent protein (GFP) and red fluorescent protein (RFP) fluorescent markers. Equal numbers of HCT116p53+/+ (green) and HCT116p53−/− (red) cells were mixed and plated in 10 cm plates, before exposure to a similar drug dosage regimen as described in FIG. 6c. At 5 days after drugs removal, the total number of HCT116p53+/+ (green) cells in the mixed population pretreated with nutlin-3 was far more than the number of HCT116p53−/− (red) cells (FIG. 6e) when compared with DMSO-treated control, which showed an equal proportion of both cell types. The mixed population treated only with nutlin-3 showed a decreased ratio of HCT116p53+/+ (green) cells to HCT116p53−/− (red) cells, perhaps' due to the inhibition of cell proliferation over the period of drug treatment.

These experiments reinforce the two key observations made in this study: (1) nutlin pretreatment renders increased survival of cells expressing wild-type p53, whereas single treatment with VX-680 abrogates proliferation of wild-type p53 cells, and (2) pretreatment with nutlin confers selective growth advantage on cells expressing wild-type p53, resulting in the preferential killing of p53-deficient cells by VX-680.

### Example 6

### Downregulation of Cyclin A2 and B1 by Gene-Specific siRNAs Suppress Endoreduplication in Response to VX-680

Consistent with a well-described role of p53 in transcriptional repression of cyclins A2 and B1 genes^{20, 40}, levels of cyclin A2 and B1 were rescued in HCT116 p53-deficient cells and A549 cells transfected with p53-specific siRNA (FIGS. 4 and 9). The rescue of expression of cyclins A2 and B1 in p53^{-/-} and p21^{-/-} cells correlates to increased polyploidy.

Next, we questioned if the depletion of cyclins A2 and B1 could mimic the observed repression of cyclins A2 and B1 and prevent endoreduplication in cells with attenuated p53 levels. Downregulation of endogenous cyclin A2 and cyclin B1 in A549 cells was achieved using siRNAs and confirmed by western blot (FIG. 10B). A549 cells were co-transfected with p53 siRNA in combination with either siRNA targeting cyclin A2 or cyclin B1 before treatment with VX-680 for 48 hours. Co-transfection of cyclin A2 siRNA reduced the percentage of polyploidy and no cells containing 16n DNA content was detected, in contrast to cells transfected only with p53 siRNA (FIG. 10A). Similarly, downregulation of cyclin B1 using gene-specific siRNA suppressed endoreduplication in p53siRNA-transfected A549. Repression of cyclin A2 and B1 genes contributes to the observed arrest in VX-680-treated since siRNA-mediated downregulation of cyclin A2 and B1 suppresses further endoreduplication in A549 cells that are transfected with p53 siRNA and exposed to VX-680.

### Example 7

### Ectopic Expression of p53 and p21 Rescues p53-Deficient Cells from Endoreduplication

p53, p21 and MDM2 proteins accumulate as early as 16 hours post drug treatment, which precedes the downregulation of cyclins A2 and B1 (FIG. 2C). This was accompanied by an increase in hypophosphorylated Rb.

To determine the mechanism by which p53 regulates the G2 checkpoint in response to aurora kinase inhibition, we used i) parental wildtype HCT116 and two derivative cell lines in which either p53 (HCT116p53^{-/-}) or p21 (HCT116p21^{-/-}) was disrupted, and ii) A549 control cells and A549 cells transfected with p53-specific siRNA. Downregulation of p53 expression abrogates the observed G1 and G2 checkpoint arrest in A549 cells exposed to VX-680, resulting in an accumulation of >15% of cells with 16n DNA content (FIG. 9). Similarly, checkpoint arrest is overridden in HCT116p53^{-/-} and HCT116p21^{-/-} (FIG. 6A and data not shown). Conversely, expression of a siRNA-resistant p53 mRNA reduced the percentage of polyploidy in A549 cells treated with p53siRNA (FIG. 4A).

To demonstrate directly if p21 alone can rescue polyploidy in p53-deficient cells, p21 was ectopically expressed in HCT116 p53^{-/-} using an Ad-CMV-p21 construct. This reduced the overall polyploid numbers to an extent similar to wildtype HCT116 which showed a predominant 8n population (FIGS. 1, 4B and 6A).

### Example 8

### VX-680 Induces an ATM-Independent G2 Checkpoint

We questioned if the observed G2 arrest is dependent on a DNA damage checkpoint. Given that aurora kinase inhibition led to an increase in the frequency of micronuclei (see Example 10 and FIG. 12), a convenient marker of chromosomal damage, we assessed if an ATM-damage dependent checkpoint is triggered in response to VX-680. ATM plays a crucial role in the activation of G2 checkpoint arrest in response to DNA-damaging agents such as ionizing radiation and etoposide.

We asked if the G2 arrest observed in VX-680-treated cells is ATM-dependent. Low concentrations of VX-680 did not induce activation of ATM as indicated by the undetectable levels of Ser1981 phosphorylation on ATM. Pre-treating cells with a ATM kinase specific inhibitor KU55933 before VX-680 treatment does not abrogate the observed G2 arrest either even though KU55933 clearly reduced γH2AX and ATM-S1981 phosphorylation in response to doxorubicin (FIG. 11A). Furthermore, caffeine did not override the G2 arrest induced by VX-680, as determined from the DNA FACS profiles (FIG. 11B). In addition, we observe an accumulation of p53 without detectable phosphorylation on Ser15, a key site phosphorylated by ATM in response to DNA damage. The lack of evidence for ATM activation coupled with an absence of γH2AX foci lesions suggested to us that the observed G2 arrest is independent of ATM activation and DNA damage checkpoint activation.

### Example 9

### Nutlin Suppress Apoptosis Induced by VX-680

We observed a decrease in colony numbers in p53 wildtype cells treated with VX-680 (FIG. 6C). To more directly test if VX-680 treatment decreases the overall numbers of surviving cells as a result of apoptosis, we analyzed the extent of apoptosis when cells are treated with various concentrations of VX-680. We found that both p53-positive cell lines (A375 and HCT116 p53^{+/+}) and p53-compromised cell lines (MDA-MB 468 and HCT116 p53^{-/-}) underwent apoptosis (FIG. 7A). This data reinforces the previous observation that there is no obvious correlation between p53 gene status and the outcome of aurora kinase inhibition in terms of the extent of endoduplication (FIG. 1) and apoptosis (FIG. 7A).

While it is not clear if cells die from VX-680-induced activation of p53-dependent apoptosis or mitotic catastrophy as a result of failed cellular divisions, it is likely that both mechanisms play a role, to variable extents in different cell line. Pretreatment with nutlin-3 resulted in >80% of viable cells even at higher concentrations of VX-680 (FIG. 7A). In contrast, there is no significant difference in the degree of apoptosis in p53-compromised cells pretreated with nutlin-3 (FIG. 7A). The order of drug treatment was important as reversing the order of drug addition (pretreatment with VX-680 before incubation with nutlin-3) resulted in an increase in apoptosis instead (FIG. 7B). Concurrent treatment with VX-680 and nutlin-3 also resulted in increase in apoptosis, albeit to a lesser extent than that observed with pretreatment with VX-680. This may be due to the partial protection of nutlin-3 on a subset of cells that arrested in G1.

We analyzed the protein levels of p53 and the downstream targets of p53 (p21, MDM2 and Bax) and asked if the apoptotic responses correlate to the differential induction of p53 responses. There is no apparent difference in the accumulation of p53, p21 or MDM2 under the various drug conditions (FIG. 7C). However, there was a significant 2-fold increase in the Bax/Bc1-2 ratio in the cells pretreated with VX-680 followed by nutlin-3 when compared to the untreated cells (FIG. 7C). This suggested that the reversed combination of VX-680 and nutlin-3 altered the Bax/Bc1-2 ratio and strongly synergizes towards apoptosis. Together, our results suggest that nutlin-3 can protect p53-positive cells from VX-680 induced apoptosis.

### Example 10

### Aurora Inhibition Induces Genomic Instability and Micronuclei Formation

Given that Aurora inhibition will impinge directly upon various processes in mitosis, including centrosomal functions, chromosome segregation and spindle assembly, we investigate further if VX-680-treated cells are associated with any other nuclear or chromosomal abnormalities. We examined the morphology of the nuclei by immunofluorescence microscopy. Micronuclei formation, indicative of chromosomal nondisjunction and chromosomal breakages, was present at an elevated frequency in VX-680-treated cells (FIG. 12). More than 70% of VX-680 treated A549 cells contained at least 1 micronucleus per nucleus compared to less than 10% in untreated A549 cells. VX-680-treated cells also displayed increased γH2AX staining in the micronuclei. Cells treated with nutlin-3 only or nutlin-3 in combination with VX-680 have a low frequency of micronuclei and γH2AX staining.

### REFERENCES

1. Carvajal D, Tovar C, Yang H, Vu B T, Heimbrook D C, Vassilev L T. Activation of p53 by MDM2 antagonists can protect proliferating cells from mitotic inhibitors. Cancer Res 2005; 65: 1918-1924.
2. Kranz D, Dobbelstein M. Nongenotoxic p53 activation protects cells against S-phasespecific chemotherapy. Cancer Res 2006; 66: 10274-10280.
3. Vassilev L T, Vu B T, Graves B, Carvajal D, Podlaski F, Filipovic Z et al. In vivo activation of the p53 pathway by small-molecule antagonists of MDM2. Science 2004; 303: 844-848.
4. Furuno N, den Elzen N, Pines J. Human cyclin A is required for mitosis until mid prophase. J Cell Biol 1999; 147: 295-306.
5. Fung T K, Ma H T, Poon R Y. Specialized roles of the two mitotic cyclins in somatic cells: cyclin A as an activator of M phase-promoting factor. Mol Biol Cell 2007; 18: 1861-1873.
6. Gong D, Pomerening J R, Myers J W, Gustaysson C, Jones J T, Hahn A T et al. Cyclin A2 regulates nuclear-envelope breakdown and the nuclear accumulation of cyclin B1. Curr Biol 2007; 17: 85-91.
7. Blasina A, Paegle E S, McGowan C H. The role of inhibitory phosphorylation of CDC2 following. DNA replication block and radiation-induced damage in human cells. Mol Biol Cell 1997; 8: 1013-1023.
8. Muller G A, Engeland K. The central role of CDE/CHR promoter elements in the regulation of cell cycle-dependent gene transcription. FEBS J 2010; 4: 877-893.
9. Yun J, Chae H D, Choy H E, Chung J, Yoo H S, Han M H et al. p53 negatively regulates cdc2 transcription via the CCAAT-binding NF-Y transcription factor. J Biol Chem 1999; 274: 29677-29682.
10. Innocente S A, Abrahamson J L, Cogswell J P, Lee J M. p53 regulates a G2 checkpoint through cyclin B1. Proc Natl Acad Sci USA 1999; 96: 2147-2152.
11. Wiman K G. Strategies for therapeutic targeting of the p53 pathway in cancer. Cell Death Differ 2006; 13: 921-926.
12. Berthet C, Aleem E, Coppola V, Tessarollo L, Kaldis P. Cdk2 knockout mice are viable. Curr Biol 2003; 13: 1775-1785.
13. Yu J, Zhang L, Hwang P M, Rago C, Kinzler K W, Vogelstein B. Identification and classification of p53-regulated genes. Proc Natl Acad Sci USA 1999; 96: 14517-14522.
14. Cheok C F, Dey A, Lane D P. Cyclin-dependent kinase inhibitors sensitize tumor cells to nutlin-induced apoptosis: a potent drug combination. Mol Cancer Res 2007; 5: 1133-1145.

It will be apparent that various other modifications and adaptations of the invention will be apparent to the person skilled in the art after reading the foregoing disclosure without departing from the spirit and scope of the invention and it is intended that all such modifications and adaptations come within the scope of the appended claims.

1. A method of selectively targeting a p53-deficient cancer cell, comprising administering to a patient suffering from cancer:
(i) a reversible cell cycle arrest-inducing agent for inducing cell cycle arrest in a p53-positive cell; and
(ii) an aurora kinase inhibitor,
wherein said reversible cell cycle arrest-inducing agent is administered prior to administration of said aurora kinase inhibitor.

2. The method according to claim 1, wherein said cancer is selected from the group consisting of leukemia; lymphoma; myeloma; skin cancer such as basal cell carcinoma (BCC), squamous cell carcinoma (SCC) or melanoma; breast cancer; head and neck cancer, such as brain cancer; colorectal cancer; colon cancer; rectal cancer; lung cancer, such as non small cell lung cancer; ovarian cancer; renal cancer; prostate cancer; liver cancer; and HPV-associated cancer such as cervical cancer.

3. The method according to claim 1, wherein said reversible cell cycle arrest-inducing agent is selected from the group consisting of nutlin-1, nutlin-2, nutlin-3 and nutlin 3a.

4. The method according to claim 1, wherein said aurora kinase inhibitor is selected from the group consisting of VX-680, AZD1152, ZM44739 and Hesperadin.

5. The method according to claim 1, wherein said reversible cell cycle arrest-inducing agent is nutlin-3 and said aurora kinase inhibitor is VX-680.

6. The method according to claim 1, wherein said reversible cell cycle arrest-inducing agent is administered 12, 16, 18, 24, 36, 48 or 72 hours prior to administration of said aurora kinase inhibitor.

7. (canceled)

8. (canceled)

9. (canceled)

10. (canceled)

11. (canceled)

12. A method of identifying a cancer patient that is likely or not to respond to a therapy, comprising determining whether a cell or tissue sample isolated from the patient is p53-positive or p53 deficient, wherein determination that the cell or tissue sample is p53 deficient identifies the patient as likely to respond to the therapy, and wherein the therapy comprises administering to a patient:
(i) a reversible cell cycle arrest-inducing agent; and
(ii) an aurora kinase inhibitor,
and wherein said reversible cell cycle arrest-inducing agent is administered prior to administration of said aurora kinase inhibitor.

13. A method of treating a cancer patient identified as likely to respond to a therapy, wherein the patient is identified by a method comprising determining whether a cell or tissue sample isolated from the patient is p53-positive or p53 deficient, wherein determination that the cell or tissue sample is p53 deficient identifies the patient as likely to respond to the therapy, and wherein the method of treatment comprises administering to the patient:
(i) a reversible cell cycle arrest-inducing agent; and
(ii) an aurora kinase inhibitor,
wherein said reversible cell cycle arrest-inducing agent is administered prior to administration of said aurora kinase inhibitor.

14. A kit for use in selectively targeting p53-deficient cancer cells, in a patient suffering from cancer, the kit comprising:
i) a reversible cell cycle arrest-inducing agent for inducing cell cycle arrest in a p53-positive cell; and
ii) an aurora kinase inhibitor,
iii) instructions to administer said reversible cell cycle arrest-inducing agent prior to administration of said aurora kinase inhibitor.

15. A method for screening to identify a compound that selectively targets a first cell type or a second cell type, wherein the first cell type is a p53-deficient cell and wherein the second cell type is a p53-positive cell, and wherein the first cell type is labelled with a first detectable marker and wherein the second cell type is labelled with a second detectable marker, the method comprising:
(i) contacting said first and second cell types with the compound; and
(ii) determining the relative amounts of the first and second detectable markers, wherein the first and second detectable marker are independently detectable and wherein a relative increase in the amount of the first marker in comparison to the amount of the second marker is indicative of a compound selectively targeting a p53-positive cell and wherein a relative increase in the amount of the second marker is indicative of a compound selectively targeting a p53-deficient cell.

16. The method according to claim 15, wherein said detectable marker is selected from the group consisting of a fluorescent compound, a radioisotope compound, a non-radioisotope compound, a bioluminescent compound, a chemiluminescent compound, a metal chelator compound, a chromogenic compound, an X-radiographic compound, and an enzyme.

17. The method according to claim 16, wherein said fluorescent compound is selected from the group consisting of a Green Fluorescent Protein (GFP), Red Fluorescent Protein (RFP), fluorescein, rhodamine, phycoerytherin, phycocyanin, allophycocyanin, o-phthaldehyde and fluorescamine.

18. The method according to claim 17, wherein said first cell type is labeled with RFP and said second cell type is labeled with GFP.

19. The method according to claim 15, wherein said cell types are contacted with the compound in vitro, ex vivo or in vivo.

20. The method of claim 15, wherein said relative amounts of the first and second marker are determined using fluorescence-activated cell sorting (FACS) or quantitative imaging microscopy.

21. An oral dosage form comprising
(i) a first composition comprising a reversible cell cycle arrest-inducing agent for inducing cell cycle arrest in a p53-positive cell; and
(ii) a second composition comprising an aurora kinase inhibitor,
wherein said first composition is formulated for immediate release on contact with aqueous media and wherein said second composition is formulated for modified release on contact with aqueous media.

22. The oral dosage form as claimed in claim 21, wherein the first and second compositions are multiparticulates.

23. The oral dosage form as claimed in claim 22, wherein the multiparticulates comprising the first composition are uncoated and wherein the multiparticulates comprising the second composition have an enteric coat.

24. The oral dosage form as claimed in 22, wherein the multiparticulates are filled into a capsule.

25. The oral dosage form as claimed in claim 21 which takes the form of a tablet in which the first and second compositions are arranged in two or more separate layers.

26. The oral dosage form as claimed in claim 21, wherein the modified release comprises delayed and/or sustained release.

27. The oral dosage form as claimed in claim 21 in which the second composition is coated with an enteric coat.
